(19) United States
(12) Patent Application Publication
(10) Pub. No.: US 2006/0078550 A1
(43) Pub. Date: Apr. 13, 2006

(54) Porcine fgl2

(76) Inventors: Gary Levy, Thornhill (CA); Anand Ghanekar, Unionville (CA); David R Grant, Toronto (CA)

Correspondence Address: BERESKIN AND PARR, 40 KING STREET WEST, BOX 401, TORONTO, ON, M5H 3Y2, CA

(21) Appl. No.: 10/502,041

(22) PCT Filed: Feb. 7, 2003
(86) PCT No.: PCT/CA03/00153

§ 371 Date: Nov. 7, 2005

Related U.S. Application Data
(60) Provisional application No. 60/354,294, filed on Feb. 7, 2002.
(60) Provisional application No. 60/355,795, filed on Feb. 12, 2002.

(51) Int. Cl. A61K 38/54; C12P 21/06; A61K 39/395; C12N 9/99; C07H 21/04; A61K 38/18

(52) U.S. Cl. 424/94.2; 435/184; 435/69.1; 435/320.1; 435/325; 530/350; 536/23.2; 424/145.1; 530/388.25

(57) ABSTRACT

The present invention relates to porcine fgl2 gene and protein and methods of modulating the gene and protein. The methods are useful in preventing thrombosis associated with the xenotransplantation of porcine organs or tissues.

### RELATED APPLICATIONS

This application claims the benefit of priority from U.S. provisional patent applications Ser. Nos. 60/354,294 filed Feb. 7, 2002 and 60/355,795 filed Feb. 12, 2002, both of which are incorporated herein by reference in their entirety.

### FIELD OF THE INVENTION

The present invention relates to novel porcine fgl2 nucleic acid and protein molecules as well as methods and compositions for immune modulation using the novel molecules.

### BACKGROUND OF THE INVENTION

Transplantation has become the treatment of choice for end-stage organ failure. Despite increasing demand, low rates of donation have resulted in a chronic shortage of available organs (1). Xenotransplantation offers one potential solution to this problem. The pig has been identified as the most suitable donor of organs for use in humans for anatomical, physiological, and ethical reasons (2).

Barriers to Xenotransplantation. Pig organs that are transplanted into nonhuman primates are destroyed by hyperacute rejection (HAR) within minutes to hours. HAR occurs due to the binding of preformed xenoreactive antibodies (XNA) to the graft endothelium, resulting in complement activation, endothelial damage, interstitial hemorrhage, thrombosis, and graft loss (3). XNA, found in humans and Old World monkeys, are directed against the galactosyl α-1,3-galactose epitope (α-gal) that is present on the cells of pigs, other lower mammals, and environmental bacteria (4). HAR has been overcome through the use of strategies aimed at inactivating or depleting XNA and complement (5,6). One of the most promising strategies has been the use of transgenic donor pigs that express human complement regulatory molecules such as decay accelerating factor (hDAF) (7).

Despite using organs from transgenic pigs in combination with antibody depletion and profound immune suppression, indefinite survival of pig-to-primate solid organ xenografts has not been achieved. Xenografts are lost after days to weeks due to a poorly understood process known as delayed xenograft rejection (DXR) (8). DXR has been associated with clinically evident abnormalities in coagulation in preclinical studies of pig-to-primate solid organ xenotransplantation (9). Thrombosis and microangiopathy are the major pathological features observed in rejected grafts; cellular infiltration is a less prominent feature. Mounting evidence suggests that complement components, coagulation factors, thromboregulatory pathways, leukocytes, cytokines, and antibodies may all play important roles in the pathogenesis of DXR (10).

Role of the endothelium in xenograft thrombosis. Elucidating the processes that contribute to thrombosis after xenotransplantation is, therefore, a principal focus of ongoing research efforts. The vascular endothelium is critically involved in regulating coagulation, and is the initial site of interaction between the xenograft and the recipient. Activation of endothelial cells (EC) in the context of xenograft rejection has been identified both in vivo and in vitro (11). “Type I activation” of EC occurs rapidly and is independent of protein synthesis. Events that occur include acute changes in cell morphology, such as shape change and retraction; release of substances such as heparan sulfate, von Willebrand factor, and tissue plasminogen activator; surface expression of molecules such as P-selectin and platelet activating factor; and loss of anticoagulant proteins such as thrombomodulin (12). “Type II activation” of EC involves sustained phenotypic alterations that depend upon increased or de novo synthesis of proteins such as adhesion molecules (eg. VCAM-1), proinflammatory cytokines (eg. IL-1), and procoagulant molecules (eg. tissue factor) (12). A wide range of xenogeneic stimuli have been observed to result in Type II activation of porcine vascular endothelial cells. These include human immunoglobulin (predominantly anti-α-gal) (13), complement (14-16), leukocytes (T lymphocytes, natural killer (NK) cells, macrophages, monocytes) (17-19), platelets (20), cytokines (IL-1, TNF-α, LPS) (21-23), and coagulation factors such as thrombin and Factor Xa (24,25).

Activated endothelial cells may directly contribute to the generation of a prothrombotic state within a solid organ xenograft. After activation by NK cells in vitro, porcine EC demonstrate an induction of procoagulant function (26). More specifically, two groups have independently demonstrated that cultured porcine aortic endothelial cells (PAEC) directly cleave human prothrombin to thrombin by an unidentified mechanism, in the absence of the classical prothrombinase complex (27,28). The production of thrombin, a central mediator of coagulation and inflammation, appears to play an important role in xenotransplantation; in vivo inhibition of thrombin is associated with prolonged xenograft survival (29,30).

Fibrinogen-like protein 2 (fgl2). Fgl2 is a novel procoagulant molecule that possesses direct prothrombinase activity. It has been implicated in the thrombosis associated with viral hepatitis, fetal loss syndromes, and transplant rejection. Fgl2 was initially described as a cytokine-induced procoagulant activity (PCA) in murine lymphoid cells, which were demonstrated to activate prothrombin directly to thrombin in the absence of factor VII or factor X (31). Subsequently, fulminant hepatic failure induced by murine hepatitis virus strain 3 (MHV-3) infection in susceptible mice was shown to be associated with a marked rise in monocyte PCA (32); monoclonal antibodies generated against PCA were shown to prevent mortality in these mice (33). Using these antibodies, a novel murine procoagulant was functionally cloned by screening of murine peritoneal macrophage cDNA libraries synthesized from MHV-3 infected mice (34,35). Sequence analysis of the MHV-3 induced prothrombinase revealed homology to musfiblp, a previously described gene encoding a mouse fibrinogen-like protein. Musfiblp had been originally cloned from cytotoxic T lymphocytes, and had been demonstrated to share significant homology to fibrinogen β and γ chains (36,37). The mRNA transcript encoding the human homologue of this molecule was subsequently isolated from T-lymphocytes, and was termed fibroleukin due to its homology with fibrinogen (38,39). The human gene encoding fibroleukin (hfgl2), was recently cloned and characterized, and studies have suggested a role for the molecule in the pathogenesis of fulminant viral hepatitis in humans (40,41). Recent experiments have also identified a principal role for fgl2 in rodent models of spontaneous abortion (42-44).

Fgl2 is highly conserved between mice and humans. The murine fgl2 (mfgl2) and hfgl2 genes localize to synthetic chromosomal loci on chromosomes 5 and 7, respectively. Comprised of two exons, both genes encode two mRNA transcripts of approximately 1.5 kb and 4.5 kb in length which are found with varying abundance in different tissues. The two variants are thought to arise on account of usage of alternative polyadenylation sequences; the longer variant contains a more lengthy 3′-untranslated region (41). The longest open reading frame encodes a protein of 432 amino acids in mice, and 439 amino acids in humans. The mfgl2 and hfgl2 proteins share 77% overall identity, and appear to share a transmembrane region near the N-terminus. The carboxy terminus of both proteins contains a highly conserved fibrinogen related domain found in the fibrinogen β and γ chains as well as other fibrinogen-like proteins (36,39). The constitutive function of fgl2 is not well understood, as the molecule has been predominantly studied in its role as an induced procoagulant. Recent experiments in our laboratory suggest that fgl2 is a membrane-bound serine protease that independently cleaves prothrombin to thrombin. Site-directed mutagenesis of serine residue 89 to alanine abolishes the prothrombinase activity. Additional experiments suggest that fgl2 may have an immunoregulatory function.

Several experiments suggest that fgl2 is implicated in allograft rejection. Elevations in factor VII independent monocyte procoagulant activity (PCA) have been shown to be associated with renal allograft rejection in humans (45,46). An increase in PCA has also been observed to correlate with small intestinal allograft rejection in rodent models (47,48). Rejection of heterotopic murine cardiac allografts has been associated with increased fgl2 expression in graft endothelial cells and infiltrating leukocytes (49). Recent experiments performed in the inventors' laboratory also support a role for fgl2 in xenograft rejection. Wild-type mouse hearts transplanted heterotopically into rats develop intravascular thrombosis and other typical features of xenograft rejection in association with increased tissue levels of fgl2 mRNA. The use of donor hearts from fgl2 knockout mice dramatically reduces the amount of thrombosis observed (50).

In view of the foregoing, there is a need in the art to clone and characterize the porcine fgl2 as a potential target for genetic modification in the pig, in order to prevent thrombosis and rejection of pig-to-primate solid organ xenografts.

### SUMMARY OF THE INVENTION

The inventors have cloned and sequenced the porcine fgl2 prothrombinase and have investigated the regulation of this molecule in porcine endothelial cells in vitro. Modulation of fgl2 expression in porcine organs or tissues that are transplanted into humans or nonhuman primates will ameliorate the thrombosis that is currently seen with pig-to-primate solid organ xenotransplantation.

Accordingly, in one aspect, the present invention provides an isolated porcine fgl2 molecule or a homolog or analog thereof. In one embodiment, the present invention provides an isolated porcine fgl2 molecule having the nucleic acid shown in FIG. 1A (SEQ ID NO:1) or a homolog or analog thereof. In another embodiment, the present invention provides an isolated porcine fgl2 molecule having the amino acid sequence found in FIG. 1B (SEQ ID NO:2) or a homolog or analog thereof.

The present invention also includes agonists and antagonists of porcine fgl2 function or activity including antisense molecules and antibodies to porcine fgl2.

The present invention includes a method of immune modulation comprising administering an effective amount of a porcine fgl2 nucleic acid or protein or an agonist or antogonist thereof to a cell or animal in need thereof.

In one aspect, the present invention provides a method of modulating an immune response by administering an effective amount of an agent that inhibits the activity of porcine fgl2.

An agent that inhibits the interaction of the porcine fgl2 protein may be an antibody that binds to the porcine fgl2 protein. Accordingly, the invention includes a method of immune modulation comprising administering an effective amount of an antibody that binds to a porcine fgl2 molecule to a cell or animal in need thereof. In one embodiment of the invention, the immune modulation is immune suppression.

Such methods of immune suppression may be useful in preventing the prothrombinase activity of porcine fgl2 which would be useful when transplanting pig organs to other animals.

Accordingly, in one embodiment the present invention provides a method of preventing thrombosis associated with xenotransplant rejection of a porcine organ or tissue comprising administering an effective amount of an agent that inhibits the activity of porcine fgl2 to the porcine organ or tissue or donor. In one embodiment, the agent is an antibody that inhibits the activity of porcine fgl2. In another embodiment, the agent is an antisense molecule of the porcine fgl2 nucleic acid sequence.

In yet another aspect, the present invention includes screening methods for identifying substances which are capable of binding to the porcine fgl2 molecules described herein. In particular, the methods may be used to identify substances or agonists which are capable of binding to and augmenting or attenuating the effects of porcine fgl2. Alternatively, the methods may be used to identify substances or antagonists which are capable of binding to porcine fgl2 and which inhibit the effects or activity of porcine fgl2.

Accordingly, the invention provides a method of identifying substances which bind with a porcine fgl2 protein, comprising the steps of:

(a) reacting the porcine fgl2 protein and a test substance, under conditions which allow for formation of a complex, and

(b) assaying for complexes of the porcine fgl2 protein and the test substance, for free substance, and for non-complexed porcine fgl2 protein, wherein the presence of complexes indicates that the test substance is capable of binding the porcine fgl2 protein.

The present invention also includes the pharmaceutical compositions Comprising any of the above molecules that modulate porcine fgl2 and/or cells expressing such molecules, for use in immune modulation.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the nucleic acid sequence (FIG. 1A; SEQ ID NO:1) and amino acid sequence (FIG. 1B; SEQ ID NO:2) of the porcine fgl2 gene.

FIG. 2 shows the alignment of the fgl2 promoter between the pig (SEQ ID NO:3), human (SEQ ID NO:4) and mouse fgl2 (SEQ ID NO:5) sequences.

FIG. 3 shows the alignment of the amino acid sequence for the pig (SEQ ID NO:2), human (SEQ ID NO:6) and mouse fgl2 (SEQ ID NO:7) sequences.

FIG. 4 is a schematic showing restriction maps of 3 clones containing porcine fgl2 gene.

FIG. 5 is a Southern blot of restriction fragments using mouse fgl2 exon 1 (161 bp) and exon 2 (659 bp) probes (done 1).

FIG. 6 is a Southern blot of restriction fragments using mouse fgl2 exon 1 (161 bp) and exon 2 (659 bp) probes (done 2).

FIG. 7 is a Southern blot of restriction fragments using mouse fgl2 exon 1 (161 bp) and exon 2 (659 bp) probes (clone 3).

FIG. 8 is a schematic showing the sequencing of clone 1.

FIG. 9 shows the 5′ and 3′ RACE data for the fgl2 gene.

FIG. 10 is a schematic showing the structure of the porcine fgl2 gene and mRNA transcripts.

FIG. 11 is a Northern blot showing fgl2 mRNA in porcine tissues.

FIG. 12 is an immunoblot showing the 3′ cleavage/poly-adenylation site of pfgl2 from a ribonuclease protection assay.

FIG. 13 is a Western blot showing the expression of recombinant pfgl2 in high five cell lysates.

FIG. 14 is a graph showing the thrombin generation by pfgl2bv-infected cell lysates.

FIG. 15 is a graph showing thrombin standard curves.

FIG. 16 is a Northern blot showing the induction of pfgl2 mRNA in PAEC.

FIG. 17 shows the chromosomal location of porcine fgl2 gene by FISH. Panel A shows an example of FISH mapping of the porcine fgl2 gene. FISH signals are localized to one porcine chromosome (arrow, left). Staining of the same mitotic figure with DAPI demonstrates that the signals are localized to chromosome 9 (right). Panel B is a schematic showing the localization of the porcine fgl2 gene to porcine chromosome 9, region q16q17. Each dot represents one pair of FISH signals detected from one out of ten images analyzed.

FIG. 18 is a Western blot showing the detection of recombinant pfgl2 protein by polyclonal rabbit anti-pfgl2 peptide antibodies.

FIG. 19 shows fgl2 −/− donor heart at 59 days post implantation in rat with normal histology (Panel A) using CsA treatment 10 mg/kg/day. Next two panels (panels B and C) show effect of withdrawing immunosuppression on day 60 with marked cellular rejection looking like allo rejection not xeno rejection. No evidence of vascular thrombosis or hemorrhage

FIG. 20 shows fgl2 +/+ heart implanted into rat with thrombosis and hemorrhage (Panels A and B). In contrast impantation of of heart from fgl2 −/− mouse (panels C and D) shows no vascular thrombosis but rather cellular rejection when no immunosuppression is used.

### DETAILED DESCRIPTION OF THE INVENTION

I. Porcine fgl2

As hereinbefore mentioned, the present inventors have isolated, cloned and sequenced the porcine fgl2 gene.

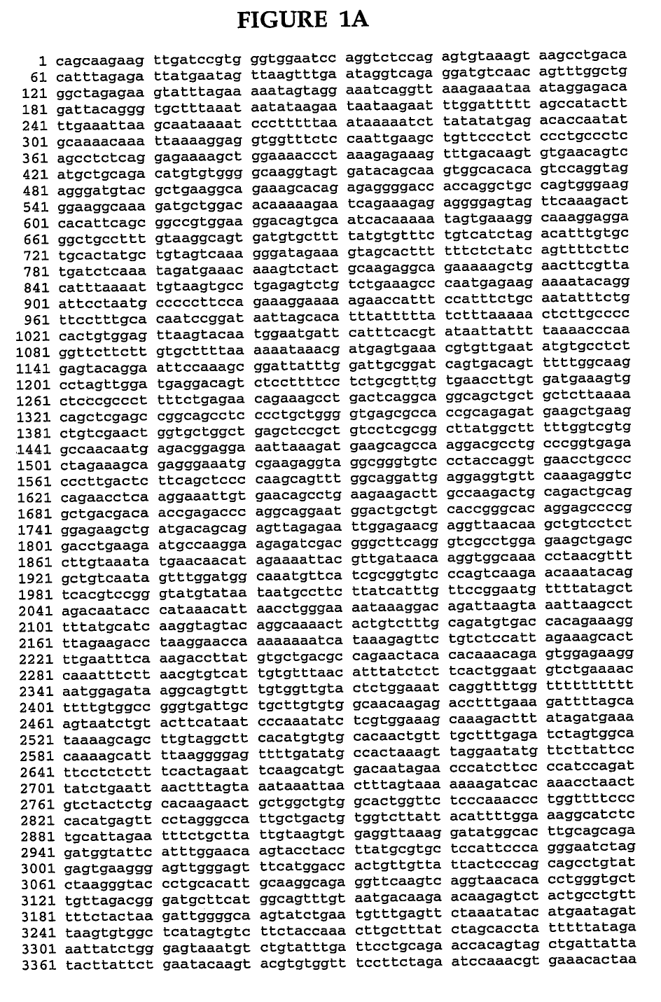

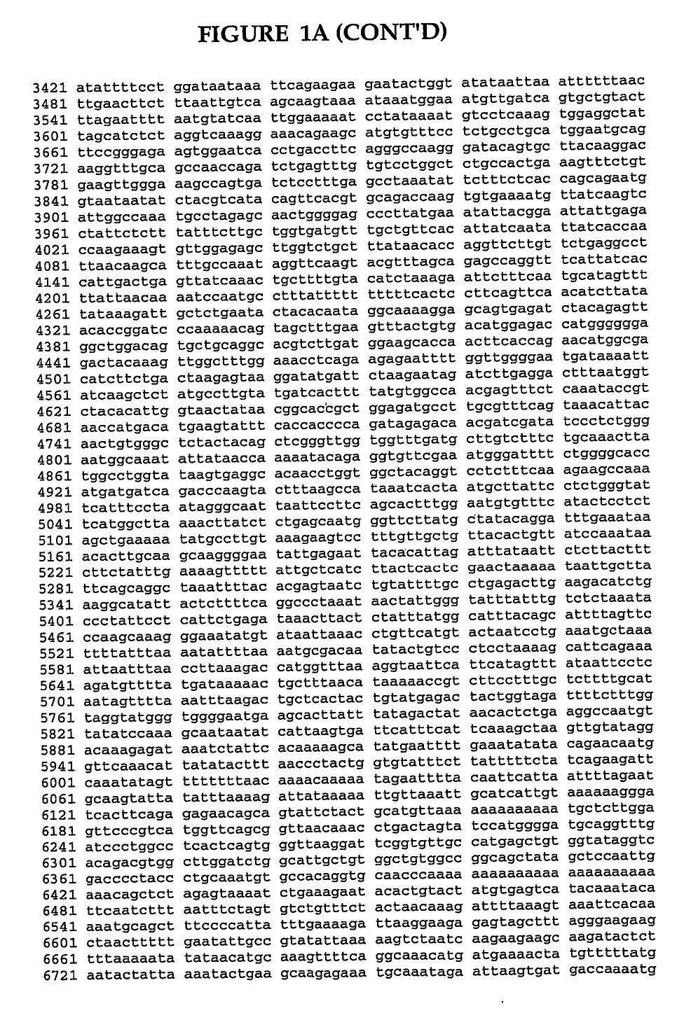

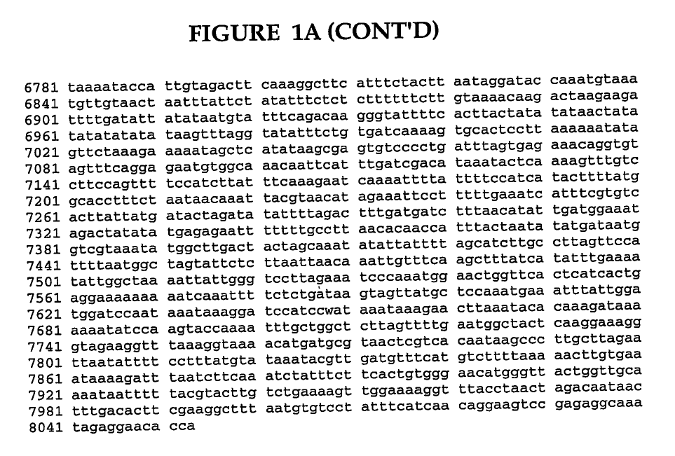

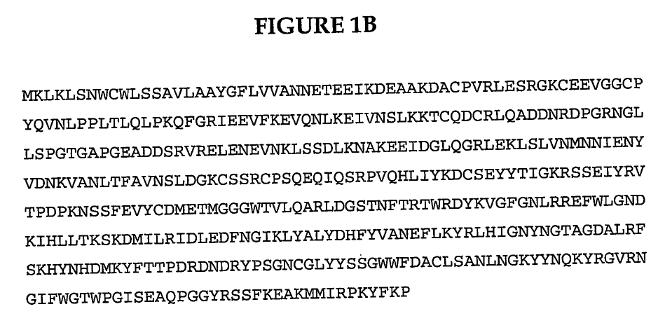

Accordingly, the present invention provides an isolated porcine fgl2 having a nucleic acid sequence shown in FIG. 1A (SEQ ID NO:1), or a homolog or analog thereof. The present invention also provides an isolated porcine fgl2 having a nucleic acid sequence that encodes an fgl2 protein having an amino acid sequence shown in FIG. 1B (SEQ ID NO:2)

The term “isolated” refers to a nucleic acid substantially free of cellular material or culture medium when produced by recombinant DNA techniques or chemical precursors or other chemicals when chemically synthesized.

The term “nucleic acid sequence” refers to a sequence of nucleotide or nucleoside monomers consisting of naturally occurring bases, sugars and intersugar (backbone) linkages. The term also includes modified or substituted sequences comprising non-naturally occurring monomers or portions thereof, which function similarly. The nucleic acid sequences of the present invention may be ribonucleic (RNA) or deoxyribonucleic acids (DNA) and may contain naturally occurring bases including adenine, guanine, cytosine, thymidine and uracil. The sequences may also contain modified bases such as xanthine, hypoxanthine, 2-aminoadenine, 6-methyl, 2-propyl, and other alkyl adenines, 5-halo uracil, 5-halo cytosine, 6-aza uracil, 6-aza cytosine and 6-aza thymine, pseudo uracil, 4-thiouracil, 8-halo adenine, 8-amino adenine, 8-thiol adenine, 8-thio-alkyl adenines, 8-hydroxyl adenine and other 8-substituted adenines, 8-halo guanines, 8-amino guanine, 8-thiol guanine, 8-thioalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

In a preferred embodiment, the porcine fgl2 nucleic acid sequence comprises:

(a) a nucleic acid sequence as shown in FIG. 1A (SEQ ID NO:1), wherein T can also be U;

(b) a nucleic acid sequence that is complimentary to a nucleic acid sequence of (a);

(c) a nucleic acid sequence that has substantial sequence homology to a nucleic acid sequence of (a) or (b);

(d) a nucleic acid sequence that is an analog of a nucleic acid sequence of (a), (b) or (c); or

(e) a nucleic acid sequence that hybridizes to a nucleic acid sequence of (a), (b), (c) or (d) under stringent hybridization conditions.

The term “sequence that has substantial sequence homology” means those nucleic acid sequences which have slight or inconsequential sequence variations from the sequences in (a) or (b), i.e., the sequences function in substantially the same manner. The variations may be attributable to local mutations or structural modifications. Nucleic acid sequences having substantial homology include nucleic acid sequences having at least 90%, and more preferably 95% identity with the nucleic add sequences as shown in FIG. 1A (SEQ ID NO:1).

The term “sequence that hybridizes” means a nucleic add sequence that can hybridize to a sequence of (a), (b), (c) or (d) under stringent hybridization conditions. Appropriate “stringent hybridization conditions” which promote DNA hybridization are known to those skilled in the art, or may be found in Current Protocols in Molecular Biology, John Wiley & Sons, N.Y. (1989), 6.3.1-6.3.6. For example, the following may be employed: 6.0× sodium chloride/sodium citrate (SSC) at about 45° C., followed by a wash of 2.0×SSC at 50° C. The stringency may be selected based on the conditions used in the wash step. For example, the salt concentration in the wash step can be selected from a high stringency of about 0.2×SSC at 50° C. In addition, the temperature in the wash step can be at high stringency conditions, at about 65° C.

The term “a nucleic add sequence which is an analog” means a nucleic acid sequence which has been modified as compared to the sequence of (a), (b) or (c) wherein the modification does not alter the function of the sequence as described herein (e.g. the analog will have fgl2 function or activity). The modified sequence or analog may have improved properties over the sequence shown in (a), (b) or (c). One example of a modification to prepare an analog is to replace one of the naturally occurring bases (i.e. adenine, guanine, cytosine or thymidine) of the sequence shown in FIG. 1A (SEQ ID NO:1) with a modified base such as such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecule shown in FIG. 1A (SEQ ID NO:1). For example, the nucleic acid sequences may contain phosphorothioates, phosphotriesters, methyl phosphonates, and phosphorodithioates.

A further example of an analog of a nucleic acid molecule of the invention is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complimentary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

The present invention also includes the novel porcine fgl2 protein. Accordingly, in one embodiment, the present invention provides an isolated porcine fgl2 protein having an amino acid sequence shown in FIG. 1B (SEQ ID NO:2) or an analog, homolog or fragment thereof.

Within the context of the present invention, a protein of the invention may include various structural forms of the primary protein which retain biological activity. For example, a protein of the invention may be in the form of acidic or basic salts or in neutral form. In addition, individual amino acid residues may be modified by oxidation or reduction.

In addition to the full length amino acid sequence, the protein of the present invention may also include truncations of the protein, and analogs, and homologs of the protein and truncations thereof as described herein. Truncated proteins or fragments may comprise peptides of at least 5, preferably 10 and more preferably 15 amino acid residues of the sequence shown in FIG. 1B (SEQ ID NO:2).

The invention further provides polypeptides comprising at least one functional domain or at least one antigenic determinant of a porcine fgl2 protein.

Analogs of the protein of the invention and/or truncations thereof as described herein, may include, but are not limited to an amino acid sequence containing one or more amino add substitutions, insertions, and/or deletions. Amino add substitutions may be of a conserved or non-conserved nature. Conserved amino acid substitutions involve replacing one or more amino acids of the proteins of the invention with amino adds of similar charge, size, and/or hydrophobicity characteristics. When only conserved substitutions are made the resulting analog should be functionally equivalent. Non-conserved substitutions involve replacing one or more amino acids of the amino add sequence with one or more amino adds which possess dissimilar charge, size, and/or hydrophobicity characteristics.

One or more amino add insertions may be introduced into the amino add sequences of the invention. Amino acid insertions may consist of single amino add residues or sequential amino acids ranging from 2 to 15 amino acids in length. For example, amino acid insertions may be used to destroy target sequences so that the protein is no longer active. This procedure may be used in vivo to inhibit the activity of a protein of the invention.

Deletions may consist of the removal of one or more amino adds, or discrete portions from the amino add sequence of the porcine fgl2. The deleted amino adds may or may not be contiguous. The lower limit length of the resulting analog with a deletion mutation is about 10 amino acids, preferably 100 amino acids.

Analogs of a protein of the invention may be prepared by introducing mutations in the nucleotide sequence encoding the protein. Mutations in nucleotide sequences constructed for expression of analogs of a protein of the invention must preserve the reading frame of the coding sequences. Furthermore, the mutations will preferably not create complementary regions that could hybridize to produce secondary mRNA structures, such as loops or hairpins, which could adversely affect translation of the receptor mRNA.

Mutations may be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site specific mutagenesis procedures may be employed to provide an altered gene having particular codons altered according to the substitution, deletion, or insertion required. Deletion or truncation of a protein of the invention may also be constructed by utilizing convenient restriction endonudease sites adjacent to the desired deletion. Subsequent to restriction, overhangs may be filled in, and the DNA religated. Exemplary methods of making the alterations set forth above are disclosed by Sambrook et al (Molecular Cloning: A Laboratory Manual, 2nd Ed., Cold Spring Harbor Laboratory Press, 1989).

The proteins of the invention also include homologs of the amino acid sequence of the porcine fgl2 protein and/or truncations thereof as described herein. Such homologs are proteins whose amino acid sequences are comprised of amino acid sequences that hybridize under stringent hybridization conditions (see discussion of stringent hybridization conditions herein) with a probe used to obtain a protein of the invention. Homologs of a protein of the invention will have the same regions which are characteristic of the protein.

A homologous protein includes a protein with an amino acid sequence having at least 90%, preferably 95% identity with the amino acid sequence of the porcine fgl2 sequence.

The invention also contemplates isoforms of the proteins of the invention. An isoform contains the same number and kinds of amino acids as a protein of the invention, but the isoform has a different molecular structure. The isoforms contemplated by the present invention are those having the same properties as a protein of the invention as described herein.

The present invention also includes a protein of the invention conjugated with a selected protein, or a selectable marker protein to produce fusion proteins. For example, the porcine fgl2 sequence is inserted into a vector that contains a nucleotide sequence encoding another peptide (e.g. GST-glutathione succinyl transferase). The fusion protein is expressed and recovered from prokaryotic (e.g. bacterial or baculovirus) or eukaryotic cells. The fusion protein can then be purified by affinity chromatography based upon the fusion vector sequence and the porcine fgl2 protein obtained by enzymatic cleavage of the fusion protein.

The proteins of the invention (including truncations, analogs, etc.) may be prepared using recombinant DNA methods. Accordingly, nucleic acid molecules of the present invention having a sequence which encodes a protein of the invention may be incorporated according to procedures known in the art into an appropriate expression vector which ensures good expression of the protein. Possible expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell”, means that the expression vectors contain a nucleic acid molecule of the invention and regulatory sequences, selected on the basis of the host cells to be used for expression, which are operatively linked to the nucleic acid molecule. “Operatively linked” is intended to mean that the nucleic acid is linked to regulatory sequences in a manner which allows expression of the nucleic acid.

The invention therefore contemplates a recombinant expression vector of the invention containing a nucleic acid molecule of the invention, or a fragment thereof, and the necessary regulatory sequences for the transcription and translation of the inserted protein-sequence. Suitable regulatory sequences may be derived from a variety of sources, including bacterial, fungal, or viral genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions.

The invention further provides a recombinant expression vector comprising a DNA nucleic add molecule of the invention cloned into the expression vector in an antisense orientation. That is, the DNA molecule is operatively linked to a regulatory sequence in a manner which allows for expression, by transcription of the DNA molecule, of an RNA molecule which is antisense to a nucleotide sequence of the invention. Regulatory sequences operatively linked to the antisense nucleic acid can be chosen which direct the continuous expression of the antisense RNA molecule.

The recombinant expression vectors of the invention may also contain a selectable marker gene which facilitates the selection of host cells transformed or transfected with a recombinant molecule of the invention. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of a target recombinant protein by acting as a ligand in affinity purification. For example, a proteolytic cleavage site may be added to the target recombinant protein to allow separation of the recombinant protein from the fusion moiety subsequent to purification of the fusion protein.

Recombinant expression vectors can be introduced into host cells to produce a transformed host cell. Accordingly, the invention includes a host cell comprising a recombinant expression vector of the invention. The term “transformed host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic add by, for example, electroporation or calcium-chloride mediated transformation. Nucleic add can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other such laboratory textbooks.

Suitable host cells include a wide variety of prokaryotic and eukaryotic host cells. For example, the proteins of the invention may be expressed in bacterial cells such as E. coli, Pseudomonas, Bacillus subtillus, insect cells (using baculovirus), yeast cells or mammalian cells. Other suitable host cells can be found in Goeddel, Gene Expression Technology: Methods in Enzymology 185, Academic Press, San Diego, Calif. (1991).

As an example, to produce porcine fgl2 proteins recombinantly, for example, E. coli can be used using the T7 RNA polymerase/promoter system using two plasmids or by labeling of plasmid-encoded proteins, or by expression by infection with M13 Phage mGPI-2. E. coli vectors can also be used with Phage lamba regulatory sequences, by fusion protein vectors (e.g. lacZ and trpE), by maltose-binding protein fusions, and by glutathione-S-transferase fusion proteins.

Alternatively, the porcine fgl2 protein can be expressed in insect cells sing baculoviral vectors, or in mammalian cells using vaccinia virus. For expression in mammalian cells, the cDNA sequence may be ligated to heterologous promoters, such as the simian virus (SV40) promoter in the pSV2 vector and introduced into cells, such as COS cells or CHO cells to achieve transient or long-term expression. The stable integration of the chimeric gene construct may be maintained in mammalian cells by biochemical selection, such as neomycin and mycophoenolic acid.

The porcine fgl2 DNA sequence can be altered using procedures such as restriction enzyme digestion, fill-in with DNA polymerase, deletion by exonuclease, extension by terminal deoxynucleotide transferase, ligation of synthetic or cloned DNA sequences, site-directed sequence alteration with the use of specific oligonucleotides together with PCR.

The cDNA sequence or portions thereof, or a mini gene consisting of a cDNA with an intron and its own promoter, is introduced into eukaryotic expression vectors by conventional techniques. These vectors permit the transcription of the cDNA in eukaryotic cells by providing regulatory sequences that initiate and enhance the transcription of the cDNA and ensure its proper splicing and polyadenylation. The endogenous porcine fgl2 gene promoter can also be used. Different promoters within vectors have different activities which alters the level of expression of the cDNA. In addition, certain promoters can also modulate function such as the glucocorticoid-responsive promoter from the mouse mammary tumor virus.

Some of the vectors listed contain selectable markers or neo bacterial genes that permit isolation of cells by chemical selection. Stable long-term vectors can be maintained in cells as episomal, freely replicating entities by using regulatory elements of viruses. Cell lines can also be produced which have integrated the vector into the genomic DNA. In this manner, the gene product is produced on a continuous basis.

Vectors are introduced into recipient cells by various methods including calcium phosphate, strontium phosphate, electroporation, lipofection, DEAE dextran, microinjection, or by protoplast fusion. Alternatively, the cDNA can be introduced by infection using viral vectors.

Porcine fgl2 proteins may also be isolated from porcine cells or issues in which the protein is normally expressed.

The protein may be purified by conventional purification methods known to those in the art, such as chromatography methods, high performance liquid chromatography methods or precipitation. For example, an anti-porcine fgl2 antibody (as described below) may be used to isolate a porcine fgl2 protein, which is then purified by standard methods.

The proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

II. Uses

The present invention includes all uses of the porcine fgl2 nucleic acid molecules and proteins of the invention including, but not limited to, the preparation of pfgl2 modulators (including antibodies and antisense oligonucleotides), the preparation of experimental systems to study porcine fgl2, as well as the use of porcine fgl2 nucleic acid sequences and proteins and modulators thereof in diagnostic and therapeutic applications. Some of the uses are further described below.

1. Porcine fgl2 Modulators

The isolation of the porcine fgl2 (pfgl2) molecule allows the development of agents that bind or modulate pfgl2. Agents that modulate pfgl2 include agents that inhibit the expression or activity of pfgl2 as well as agents that enhance or increase the expression of pfgl2. The agent can be any type of substance, including, but not limited to, nucleic acids (including antisense oligonucleotides), proteins (including antibodies), peptides, peptide mimetics, carbohydrates and small molecules (including organic and inorganic compounds). Some pfgl2 modulators are described below.

(a) Antibodies

The isolation of the porcine fgl2 protein enables the preparation of antibodies specific for porcine fgl2. Accordingly, the present invention provides an antibody that binds to a porcine fgl2 protein. Antibodies may be used advantageously to monitor the expression of porcine fgl2 or in therapeutic or diagnostic assays described below. Antibodies can be prepared which bind a distinct epitope in an unconserved region of the protein. An unconserved region of the protein is one that does not have substantial sequence homology to other fgl2 proteins.

Conventional methods can be used to prepare the antibodies including the method described in Example 3. For example, by using a peptide of porcine fgl2, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for porcine fgl2 as described herein.

The term “antibody” as used herein is intended to include fragments thereof which also specifically react with porcine fgl2 or peptide thereof, having the activity of the porcine fgl2. Antibodies can be fragmented using conventional techniques and the fragments screened for utility in the same manner as described above. For example, F(ab′)² fragments can be generated by treating antibody with pepsin. The resulting F(ab′)² fragment can be treated to reduce disulfide bridges to produce Fab′ fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes the gene product of porcine fgl2 antigens of the invention (See, for example, Morrison et al., Proc. Natl. Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)); and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against porcine fgl2 proteins may also be generated by screening expression libraries encoding immunoglobulin genes, or portions thereof, expressed in bacteria with peptides produced from the nucleic acid molecules of porcine fgl2. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

(b) Antisense Oligonucleotides

Isolation of a nucleic acid molecule encoding porcine fgl2 enables the production of antisense oligonucleotides that can modulate the expression and/or activity of porcine fgl2.

Accordingly, the present invention provides an antisense oligonucleotide that is complimentary to a nucleic acid sequence encoding porcine fgl2.

The term “antisense oligonucleotide” as used herein means a nucleotide sequence that is complimentary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complimentary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo. In one embodiment, the antisense oligonucleotide may be delivered to macrophages and/or endothelial cells in a liposome formulation.

(c) Peptide Mimetics

The present invention also includes peptide mimetics of the pfgl2 protein. Such peptides may include competitive inhibitors, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a pfgl2 peptide, or enhancer or inhibitor of the pfgl2 peptide. Peptide mimetics also include molecules incorporating peptides into larger molecules with other functional elements (e.g., as described in WO 99/25044). Peptide mimetics also include peptoids, oligopeptoids (Simon et al (1972) Proc. Natl. Acad, Sci USA 89:9367), and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

(d) Other Substances

In addition to the above substances, other substances that can modulate pfgl2 can also be identified and used in the methods of the invention. For example, substances which can bind pfgl2 may be identified by reacting pfgl2 with a substance which potentially binds to pfgl2, then detecting if complexes between the pfgl2 and the substance have formed. Substances that bind pfgl2 in this assay can be further assessed to determine if they are useful in modulating or inhibiting pfgl2 and useful in the therapeutic methods of the invention.

Accordingly, the present invention also includes a method of identifying substances which can bind to pfgl2 comprising the steps of:

(a) reacting pfgl2 and a test substance, under conditions which allow for formation of a complex between the pfgl2 and the test substance, and

(b) assaying for complexes of pfgl2 and the test substance, for free substance or for non complexed pfgl2, wherein the presence of complexes indicates that the test substance is capable of binding pfgl2.

Conditions which permit the formation of substance and pfgl2 complexes may be selected having regard to factors such as the nature and amounts of the substance and the protein.

The substance-pfgl2 complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against pfgl2 or the substance, or labelled pfgl2, or a labelled substance may be utilized. The antibodies, pfgl2, or substances may be labelled with a detectable substance.

The pfgl2 or the test substance used in the method of the invention may be insolubilized. For example, the pfgl2 or substance may be bound to a suitable carrier. Examples of suitable carriers are agarose, cellulose, dextran, Sephadex, Sepharose, carboxymethyl cellulose, polystyrene, filter paper, ion-exchange resin, plastic film, plastic tube, glass beads, silica, polyamine-methyl vinyl-ether-maleic acid copolymer, amino acid copolymer, ethylene-maleic add copolymer, nylon, silk, etc. The carrier may be in the shape of, for example, a tube, test plate, beads, disc, sphere etc.

The insolubilized pfgl2 or substance may be prepared by reacting the material with a suitable insoluble carrier using known chemical or physical methods, for example, cyanogen bromide coupling.

The pfgl2 or test substance may also be expressed on the surface of a cell in the above assay.

The pfgl2 gene or protein may be used as a target for identifying lead compounds for drug development. The invention therefore includes an assay system for determining the effect of a test compound or candidate drug on the activity of the pfgl2 gene or protein.

Accordingly, the present invention provides a method for identifying a compound that modulates pfgl2 gene or protein activity comprising:

(a) incubating a test compound with a pfgl2 protein or a nucleic acid encoding a pfgl2 protein; and

(b) determining the effect of the test compound on pfgl2 protein activity or pfgl2 gene expression and comparing with a control (i.e. in the absence of a test compound) wherein a change in the pfgl2 protein activity or pfgl2 gene expression as compared to the control indicates that the test compound is a potential modulator of the pfgl2 gene or protein.

2. Experimental Systems

Eukaryotic expression systems can be used for many studies of the porcine fgl2 gene and gene product(s) including determination of proper expression and post-translational modifications for full biological activity, identifying regulatory elements located in the 5′ region of the porcine fgl2 gene and their role in tissue regulation of protein expression, production of large amounts of the normal and mutant protein for isolation and purification, to use cells expressing the porcine fgl2 protein as a functional assay system for antibodies generated against the protein or to test effectiveness of pharmacological agents.

3. Diagnostic Assays

The isolation of porcine fgl2 molecules allows the detection of these molecules in cells and organs and the diagnosis of conditions involving an increase or decrease in porcine fgl2 activity or expression. For example, the nucleic adds, proteins and/or antibodies can be used to evaluate a pig organ prior to transplantation to assess the levels of fgl2. If the organ is from a transgenic knockout pig, one would need to verify lack of expression of fgl2 prior to transplantation.

Accordingly, the present invention provides a method of detecting a porcine fgl2 protein or nucleic acid in a sample (including an absence) comprising assaying the sample for (a) a nucleic acid molecule encoding a porcine fgl2 protein or a fragment thereof or (b) a porcine fgl2 protein or a fragment thereof.

(a) Nucleic Acid Molecules

The nucleic acid molecules encoding porcine fgl2 as described herein or fragments thereof, allow those skilled in the art to construct nucleotide probes for use in the detection of nucleotide sequences encoding porcine fgl2 or fragments thereof in samples, preferably biological samples such as cells, tissues, organs and bodily fluids. The probes can be useful in detecting the presence of a condition associated with porcine fgl2 or monitoring the progress of such a condition. The probes are also useful in detecting the presence of porcine fgl2 in a pig organ prior to transplantation. Accordingly, the present invention provides a method for detecting a nucleic acid molecules encoding porcine fgl2 comprising contacting the sample with a nucleotide probe capable of hybridizing with the nucleic acid molecule to form a hybridization product, under conditions which permit the formation of the hybridization product, and assaying for the hybridization product.

Example of probes that may be used in the above method include fragments of the nucleic acid sequences shown in FIG. 1A (SEQ ID NO:1). A nucleotide probe may be labelled with a detectable substance such as a radioactive label which provides for an adequate signal and has sufficient half-life such as 32P, 3H, 14C or the like. Other detectable substances which may be used include antigens that are recognized by a specific labelled antibody, fluorescent compounds, enzymes, antibodies specific for a labelled antigen, and chemiluminescence. An appropriate label may be selected having regard to the rate of hybridization and binding of the probe to the nucleic acid to be detected and the amount of nucleic acid available for hybridization. Labelled probes may be hybridized to nucleic acids on solid supports such as nitrocellulose filters or nylon membranes as generally described in Sambrook et al, 1989, Molecular Cloning, A Laboratory Manual (2nd ed.). The nucleotide probes may be used to detect genes, preferably in human cells, that hybridize to the nucleic acid molecule of the present invention preferably, nucleic acid molecules which hybridize to the nucleic acid molecule of the invention under stringent hybridization conditions as described herein.

Nucleic acid molecules encoding a porcine fgl2 protein can be selectively amplified in a sample using the polymerase chain reaction (PCR) methods and cDNA or genomic DNA. It is possible to design synthetic oligonucleotide primers from the nucleotide sequence shown in FIG. 1A (SEQ ID NO:1) for use in PCR. A nucleic acid can be amplified from cDNA or genomic DNA using oligonucleotide primers and standard PCR amplification techniques. The amplified nucleic acid can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

(b) Proteins

The porcine fgl2 protein may be detected in a sample using antibodies that bind to the protein as described in detail above. Accordingly, the present invention provides a method for detecting a porcine fgl2 protein comprising contacting the sample with an antibody that binds to porcine fgl2 which is capable of being detected after it becomes bound to the porcine fgl2 in the sample.

Antibodies specifically reactive with porcine fgl2 or derivatives thereof, such as enzyme conjugates or labeled derivatives, may be used to detect porcine fgl2 in various biological materials, for example they may be used in any known immunoassays which rely on the binding interaction between an antigenic determinant of porcine fgl2 and the antibodies. Examples of such assays are radioimmunoassays, enzyme immunoassays (e.g. ELISA), immunofluorescence, immunoprecipitation, latex agglutination, hemagglutination and histochemical tests. Thus, the antibodies may be used to detect and quantify porcine fgl2 in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect porcine fgl2 to localise it to particular cells and tissues and to specific subcellular locations, and to quantitate the level of expression.

Cytochemical techniques known in the art for localizing antigens using light and electron microscopy may be used to detect porcine fgl2. Generally, an antibody of the invention may be labelled with a detectable substance and porcine fgl2 may be localised in tissue based upon the presence of the detectable substance. Examples of detectable substances include various enzymes, fluorescent materials, luminescent materials and radioactive materials. Examples of suitable enzymes include horseradish peroxidase, biotin, alkaline phosphatase, β-galactosidase, or acetylcholinesterase; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include radioactive iodine I-125, I-131 or 3-H. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against porcine fgl2. By way of example, if the antibody having specificity against porcine fgl2 is a rabbit IgG antibody, the second antibody may be goat anti-rabbit gamma-globulin labelled with a detectable substance as described herein.

Where a radioactive label is used as a detectable substance, porcine fgl2 may be localized by autoradiography. The results of autoradiography may be quantitated by determining the density of particles in the autoradiographs by various optical methods, or by counting the grains.

4. Therapeutic Methods

As previously stated, the present inventors have demonstrated that the porcine fgl2 molecule is a procoagulant that possesses prothrombinase activity. It is involved in the thrombosis associated with viral hepatitis, fetal loss syndromes and transplant rejection (see WO 98/51335, which is incorporated herein by reference in its entirety). As a result, the novel porcine fgl2 molecules described herein may be used in immune modulation in pigs. Consequently, the present invention includes methods of modulating an immune response caused by fgl2 using the porcine fgl2 described herein.

Accordingly, the present invention provides a method of modulating an immune response comprising administering an effective amount of a porcine fgl2 nucleic acid or protein or a modulator thereof to a porcine cell or animal in need thereof. The present invention also includes a use of an effective amount of a porcine fgl2 nucleic add or protein or a modulator thereof to modulate an immune response or to manufacture a medicament to modulate an immune response.

The term “modulate an immune response” as used herein refers to the suppression, including inducing immune tolerance, or activation of the immune response.

The term “effective amount” as used herein means an amount effective, at dosages and for periods of time necessary to achieve the desired results. Effective amounts of a molecule may vary according to factors such as the disease state, age, sex, weight of the animal. Dosage regima may be adjusted to provide the optimum therapeutic response. For example, several divided doses may be administered daily or the dose may be proportionally reduced as indicated by the exigencies of the therapeutic situation.

The immune responses that may be modulated include modulating graft rejection, fetal loss and viral infections.

In an embodiment, the present invention provides a method of suppressing or inhibiting an immune response to a transplanted porcine organ or tissue comprising administering an effective amount of an agent that inhibits porcine fgl2 to the porcine organ or tissue, porcine donor or transplant recipient. The present invention also includes a use of an effective amount of an agent that inhibits porcine fgl2 to suppress or inhibit an immune response or to manufacture a medicament to suppress or inhibit an immune response.

The porcine organ or tissue can be any organ or tissue that one wishes to transplant to a recipient including, but not limited to, heart, liver, kidney, lung, pancreas, pancreatic islets, brain tissue, cornea, bone, intestine, skin and hematopoietic cell.

In a specific application, the present invention provides a method of inhibiting or preventing thrombosis associated with xenotransplant rejection of a porcine organ or tissue comprising administering an effective amount of an agent that inhibits porcine fgl2 to the porcine organ or tissue, porcine donor or transplant recipient. The present invention also includes a use of an effective amount of an agent that inhibits porcine fgl2 to inhibit or prevent thrombosis associated with xenotransplant rejection of a porcine organ or tissue or to manufacture a medicament to inhibit or prevent thrombosis associated with xenotransplant rejection of a porcine organ or tissue.

The term “agent that inhibits porcine fgl2” includes any substance or agent that inhibits the activity of the porcine fgl2 protein or expression of the porcine fgl2 gene. The agent can be selected from any of the pfgl2 modulators described above in Section 1.

In one embodiment, the agent is an antibody that inhibits the activity of porcine fgl2. In another embodiment, the agent is an antisense molecule of the porcine fgl2 nucleic acid sequence.

In a further embodiment, the porcine fgl2 gene is inhibited by preparing a transgenic knockout pig that lacks expression of the fgl2 gene. As evidenced in Example 4, the inventors have prepared a transgenic knockout animal lacking expression of the fgl2 gene. The inventors demonstrated that cardiac xenografts from the fgl2 knockout animals did not develop thrombosis or any other features of graft rejection when transplanted into an immunocompetent rat.

Accordingly, the present invention provides a transgenic pig (or its progeny) lacking expression of the pfgl2 gene.

The present invention also provides a method of suppressing an immune response to a porcine organ or tissue comprising (a) preparing a transgenic pig lacking expression of a pfgl2 gene and (b) transplanting an organ from the transgenic pig to a recipient animal. The recipient animal is preferably a human. The animal preferably receives immunosuppressive drugs either before, during and/or after the transplant.

Transgenic pigs lacking expression of the pfgl2 gene can be prepared using techniques known in the art. For example, see U.S. Pat. No. 6,498,285, reference 56 as well as Example 5. Briefly, a nucleic acid construct is prepared that can be used to inactivate the endogenous porcine fgl2 gene. The nucleic acid construct will comprise a disrupted porcine fgl2 gene which is generally disrupted by the insertion of an exogenous sequence into the fgl2 gene such that expression of the fgl2 gene is inhibited. The exogenous sequence is generally inserted into an exon of the gene and generally comprises a selectable marker. Preferred marker genes are antibiotic resistance genes such as the neomycin resistance gene (neo), the reporter lacZ gene and the herpes simplex virus thymidine kinase gene (HSV-tk). The marker gene will preferably have a 3-UTR sequence attached to the 3′ end of the gene which serves to stabilize the marker gene. The nucleic acid construct is inserted into a porcine cell, preferably an embryonic cell such as the pro-nuclei of a fertilized egg. The transfected embryonic cells are inserted into a pseudopregnant mother.

Accordingly, the present invention includes a method for preparing a transgenic pig comprising an inactivated fgl2 gene comprising

(a) inserting into a porcine embryonic cell a nucleic acid construct comprising a disrupted porcine fgl2 gene;

(b) implanting the transfected embryonic cell into a female pig; and

(c) permitting said embryo to develop into a pig.

The transgenic pig is preferably further mated with a second transgenic pig heterozygous for the nucleic acid construct and progeny are selected that are homozygous for the nucleic acid construct. Applicants have successfully prepared a transgenic knockout fgl2 mouse as described in their co-pending U.S. application Ser. No. 09/689,872 which is incorporated herein by reference in its entirety. Similar methodology and constructs can be used to prepare a transgenic pig, for instance as described in Example 5.

5. Pharmaceutical Compositions

The present invention includes pharmaceutical compositions containing the porcine fgl2 molecules and modulators thereof of the invention. Accordingly, the present invention provides a pharmaceutical composition comprising a porcine fgl2 protein, a nucleic acid molecule encoding a porcine fgl2 protein and/or a modulator of a porcine nucleic acid sequence or protein in admixture with a suitable diluent or carrier. In one embodiment, the pharmaceutical composition comprises an effective amount of an agent that inhibits porcine fgl2 in admixture with a suitable diluent or carrier. The agent that inhibits porcine fgl2 is preferably an antibody or an antisense molecule.

Such pharmaceutical compositions can be for intralesional, intravenous, topical, rectal, parenteral, local, inhalant or subcutaneous, intradermal, intramuscular, intrathecal, transperitoneal, oral, and intracerebral use. The composition can be in liquid, solid or semisolid form, for example pills, tablets, creams, gelatin capsules, capsules, suppositories, soft gelatin capsules, gels, membranes, tubelets, solutions or suspensions. The porcine fgl2 is preferably injected in a saline solution either intravenously, intraperitoneally or subcutaneously.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. The pharmaceutical compositions may additionally contain other immune modulatory agents.

A pharmaceutical composition comprising the nucleic acid molecules of the invention may be used in gene therapy to inhibit the activity of porcine fgl2. Recombinant molecules comprising an antisense oligonucleotide may be directly introduced into cells or tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes. Recombinant molecules may also be delivered in the form of an aerosol or by lavage. The nucleic acid molecules of the invention may also be applied extracellularly such as by direct injection into cells.

The following non-limiting examples are illustrative of the present invention:

### EXAMPLES

### Example 1

Cloning of the Porcine fgl2 Gene (pfgl2)

A bacteriophage λ EMBL3 SP6/T7 adult porcine genomic library was obtained from Clontech Laboratories (Palo Alto, Calif., USA). A 659 bp PCR product was amplified from exon 2 of the mouse fgl2 (mfgl2) cDNA and labelled with α-³²P-dCTP for use as a probe. Approximately 1.2×10⁶ viral clones were screened by hybridization with the radiolabeled mfgl2 probe. Three positive clones were identified, with porcine genomic DNA inserts ranging in length between 13 and 17 kb. After plaque purification, the three clones were individually plated at high density and their DNA was extracted from the bacterial lysate.

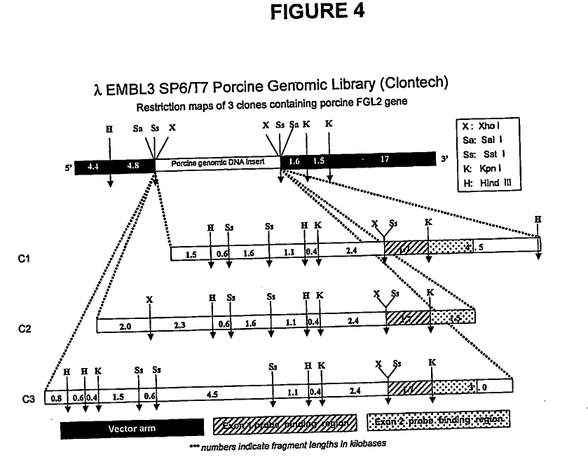

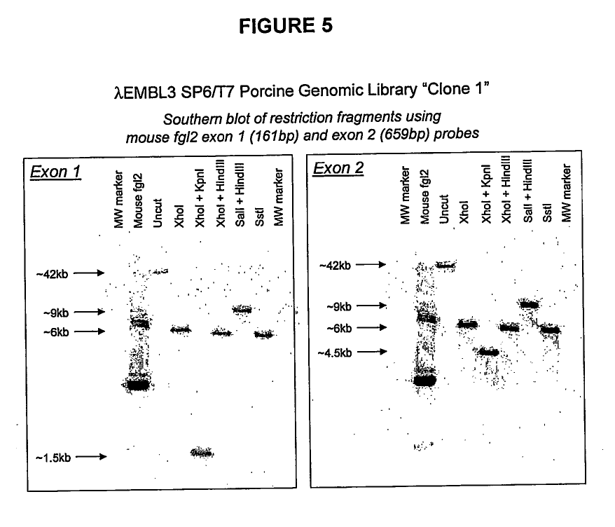

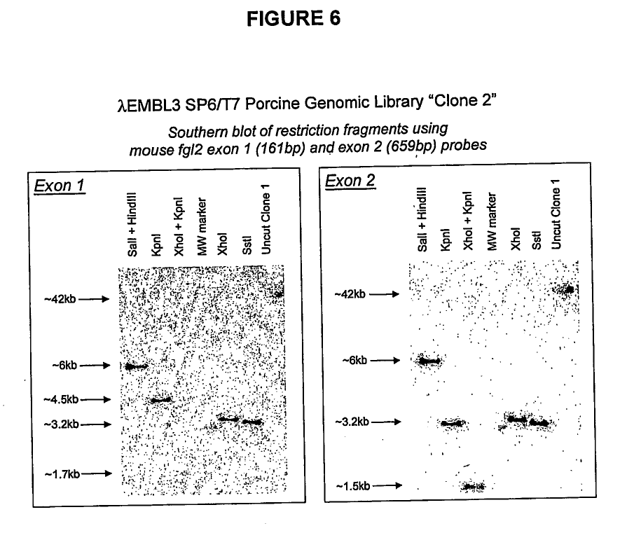

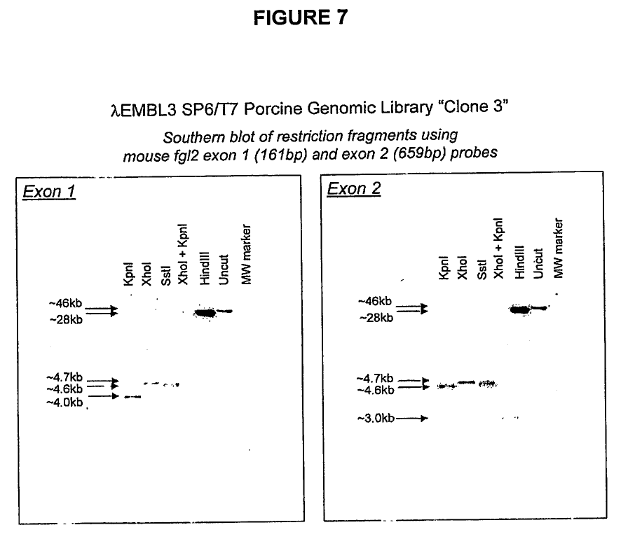

A restriction map of each clone was prepared by digestion with XhoI, SalI, SstI, HindIII, and KpnI, alone and in combination (FIG. 4). Restriction fragments from each clone that contained exons 1 and/or 2 were identified by Southern blotting using the murine exon 2 probe and a 161 bp murine exon 1 probe (FIG. 7). The pfgl2 gene was localized within each clone's porcine genomic DNA insert. The clone containing the greatest portion of the gene was selected for DNA sequencing on both strands. Approximately 8 kb of sequence data was obtained. Using porcine genomic DNA as a template, additional sequence at the 3′ end of the gene was obtained by PCR using a forward primer based on the 3′ porcine sequence obtained in combination with a reverse primer based on the human fgl2 gene sequence. The 1.2 kb PCR product overlapped with the porcine sequence obtained from the genomic library clone by ˜800 bp (100% alignment), providing ˜400 bp of additional sequence.

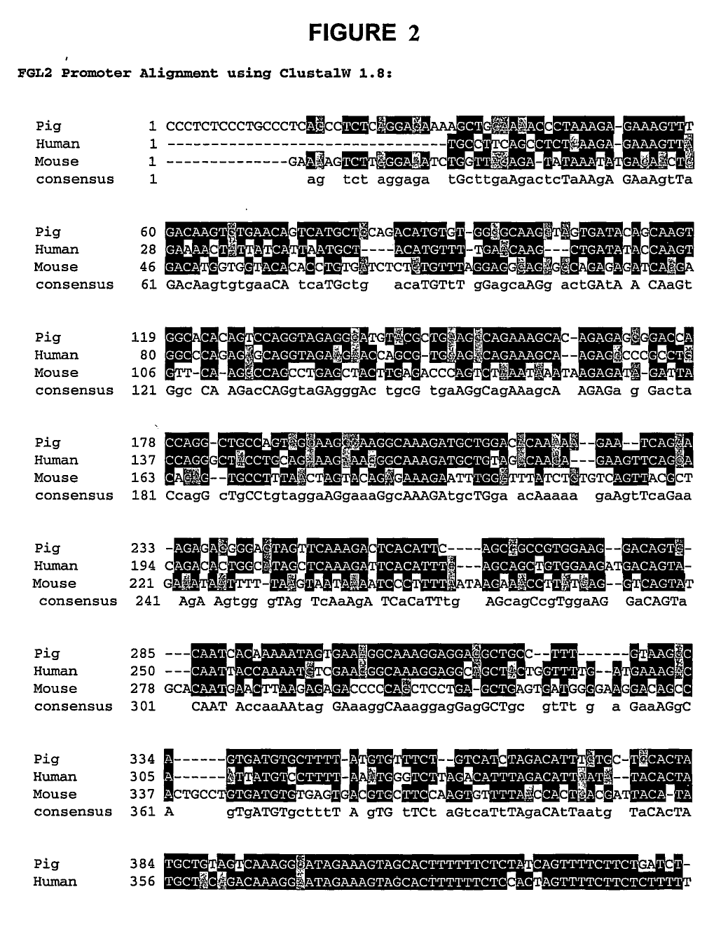

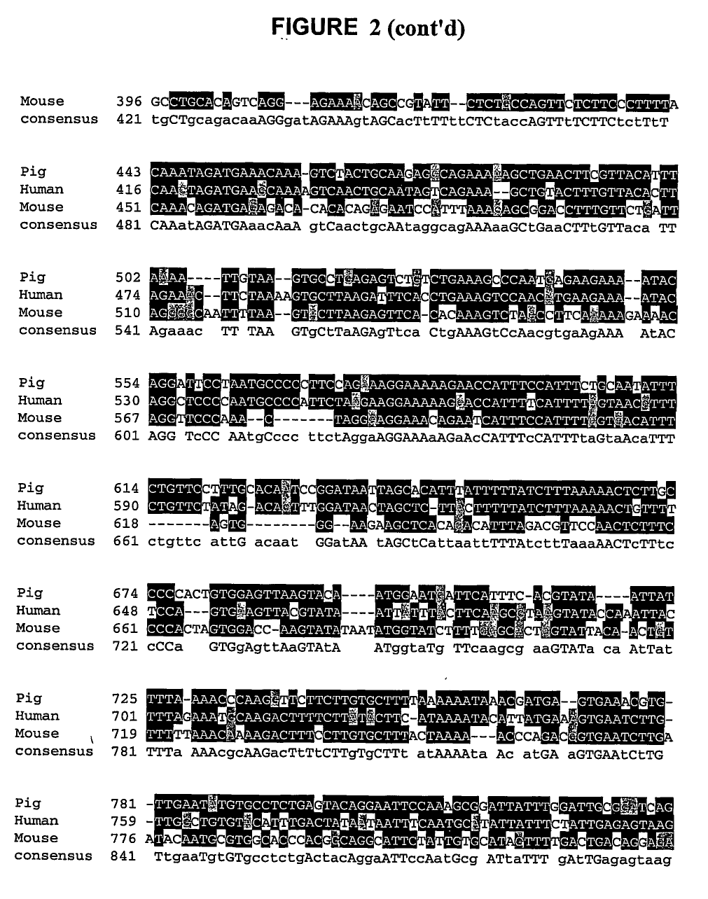

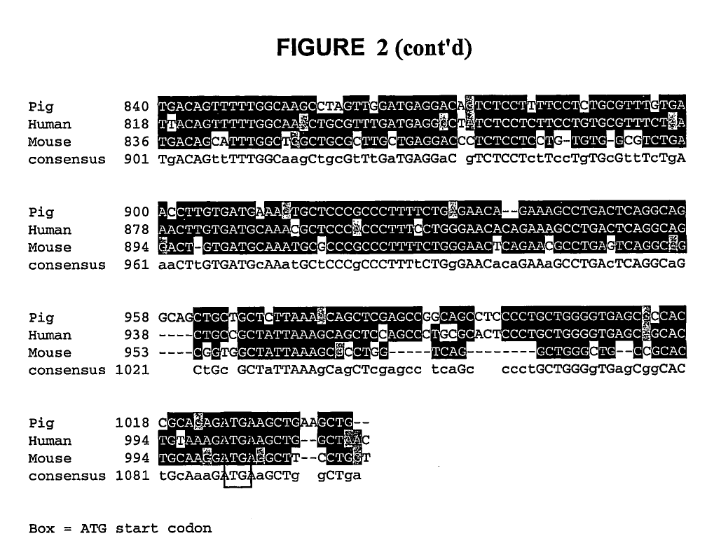

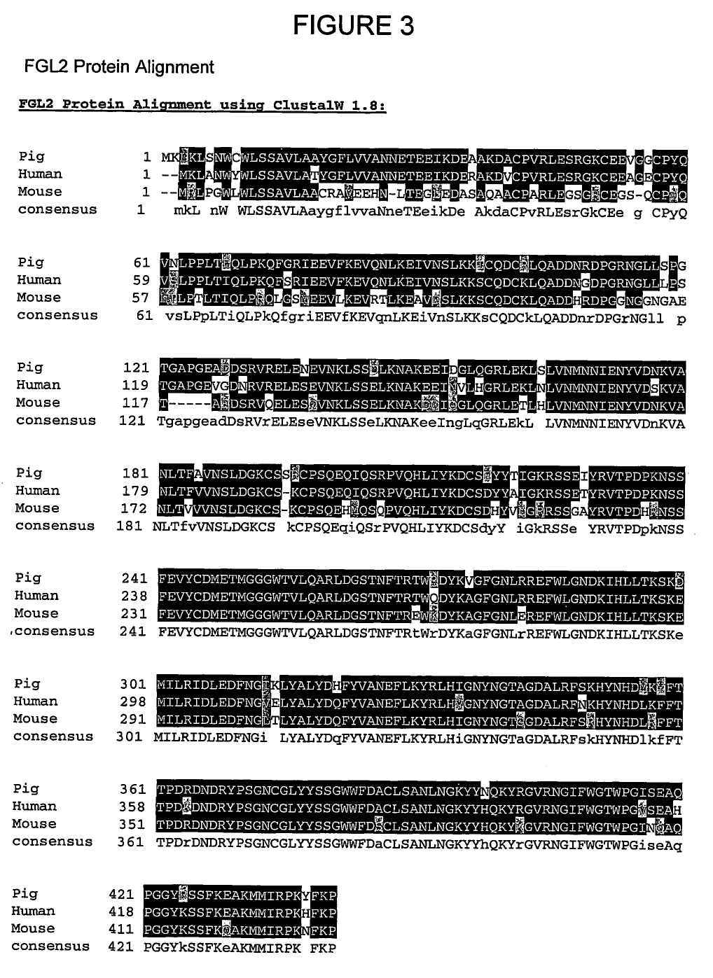

Analysis of the composite sequence data revealed significant similarity to the mfgl2 and hfgl2 genes at the nucleotide level. In addition to the two exons and one intron, the cloned region included approximately 1.3 kb of the promoter region. The complete gene sequence is provided in FIG. 1A (SEQ IN NO:1), with annotations. An alignment of the mouse, human, and porcine fgl2 promoter regions is included in FIG. 2.

Cloning of the pfgl2 cDNA

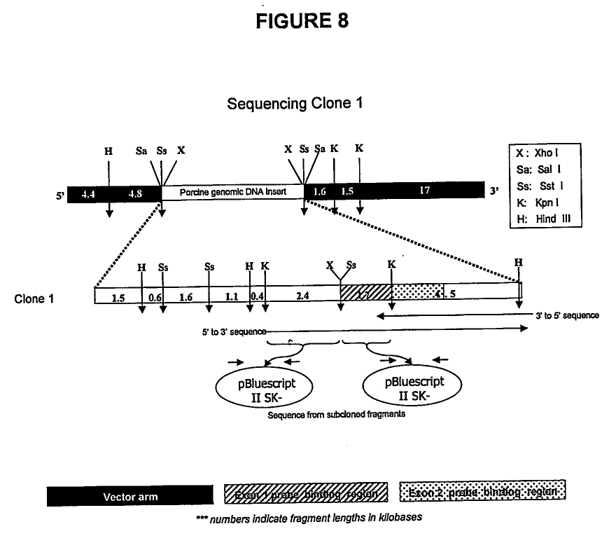

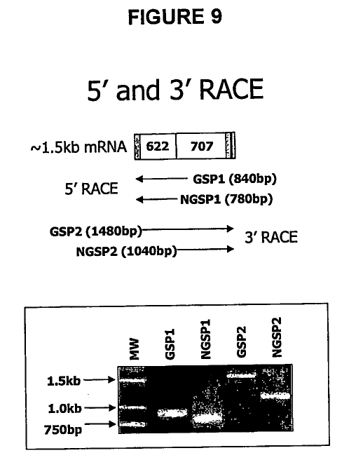

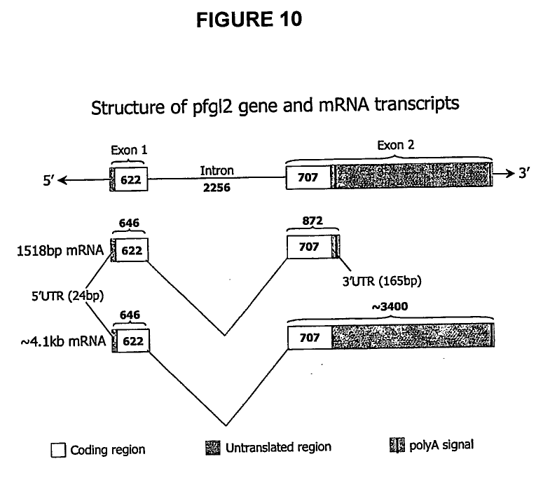

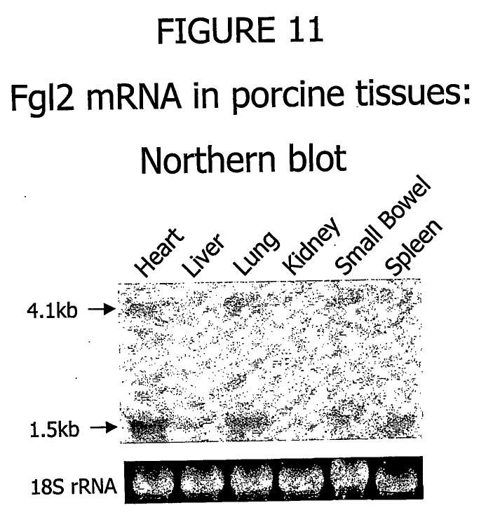

Northern blotting on porcine tissues identified a greater abundance of fgl2 in heart, lung, and small bowel, as shown in FIG. 11. Two mRNA transcripts of approximately 1.5 kb and 4.2 kb were seen by Northern analysis, corresponding to findings in other species (41). Total RNA from porcine small intestine was used for 5′ and 3′ RACE (rapid amplification of cDNA ends) experiments and ribonuclease protection assays (RPA) in order to identify the transcription start site and the 3′ cleavage/polyadenylation sites of the two mRNA species. The findings of these studies is summarized in FIGS. 8 and 9 and in the sequence in FIG. 1A (SEQ ID NO:1). 5′ RACE identified a transcription start site 24 nucleotides upstream of the start codon (ATG). 3′ RACE identified a 3′ mRNA cleavage/polyadenylation site 165 nucleotides downstream of the predicted stop codon, at a position 17 nucleotides past the predicted polyadenylation signal for the short mRNA transcript. RACE therefore predicts a cDNA of 1518 nucleotides corresponding to the size of the short mRNA transcript seen on Northern blot.

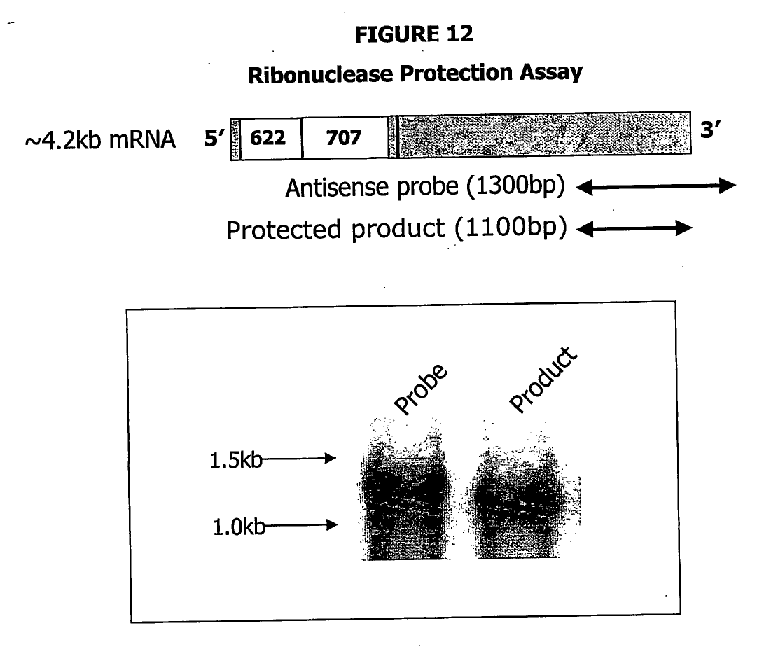

A 3′ cleavage/polyadenylation site corresponding to the longer mRNA variant was not identified by 3′ RACE. The 3′ UTR of the pfgl2 gene contains a 27 nucleotide stretch of adenine residues which may have acted as a priming site for first-strand cDNA synthesis, preventing the synthesis of cDNA corresponding to the downstream sequence of the longer mRNA transcript. Ribonuclease protection assays were therefore utilized to identify the longer mRNA transcript. Genomic DNA sequence surrounding the region of the predicted distal 3′ cleavage/polyadenylation site was amplified by PCR and subcloned into the multiple cloning site of a vector flanked by Sp6 and T7 promoters for in vitro transcription. Digoxigenin (Dig)-labelled sense and antisense RNA probes were synthesized by performing in vitro transcription with either T7 or Sp6 RNA polymerases in the presence of a dNTP pool containing Dig-dUTP. Sense and antisense probes were hybridized against 100 mg of porcine small intestinal total RNA, digested with RNase, and then resolved by denaturing PAGE. The RNA in the gel was transferred to a positively charged nylon membrane and subsequently detected by chemiluminescence after immunoblotting with an anti-dig antibody (Roche). The length of protected RNA fragment corresponded to the predicted second 3′ cleavage/polyadenylation site (FIG. 12).

Analysis of the pfgl2 mRNA sequence revealed an open reading frame (ORF) that encodes a 442 amino acid protein (FIG. 1B, SEQ ID NO:2) which shares 89% overall homology to hfgl2 and 77% overall homology to mfgl2. The putative N-terminal transmembrane region, the region surrounding the prothrombinase active site (serine 89 in mouse), and the C-terminal fibrinogen-related domain are very highly conserved. The coding region is provided in FIG. 1A (SEQ ID NO:1). An alignment of the porcine, human, and mouse fgl2 protein sequences is provided in FIG. 1B (SEQ ID NO:2).

Expression of Recombinant pfgl2 Protein

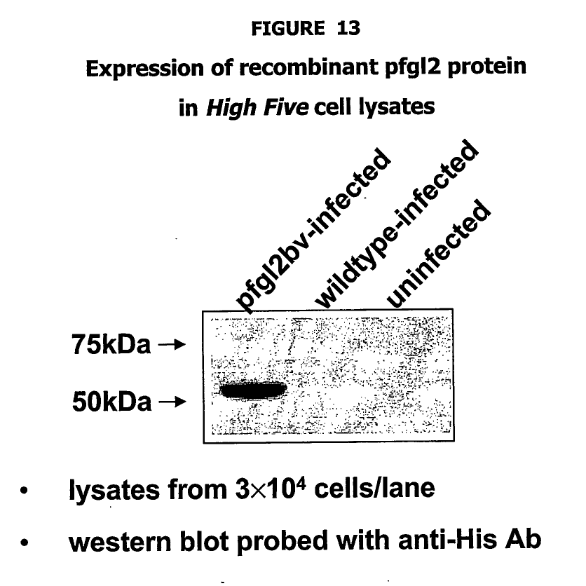

The predicted full-length coding region of pfgl2 (including the stop codon) was amplified by RT-PCR from small intestinal total RNA and subcloned into the baculovirus transfer vector pAcHLT-C (BD Pharmingen), in-frame with an N-terminal polyhistidine (His) tag. Spodoptera frugiperda 9 (Sf9) insect cells were co-transfected with pAcHLT-C/pfgl2 and linearized BaculoGold baculovirus DNA (BD Pharmingen) in order to generate a recombinant pfgl2-encoding baculovirus (pfgl2bv) by homologous recombination. The recombinant virus was plaque purified, amplified, and titered. High-Five insect cells were infected at a multiplicity of infection (MOI) of 3. Western blotting of the cell pellet with an anti-His antibody was used to demonstrate expression of the 55 kilodalton (kDa) recombinant His-tagged pfgl2 cell lysates harvested after three days of infection (FIG. 13).

Generation of Thrombin From Prothrombin by Recombinant pfgl2

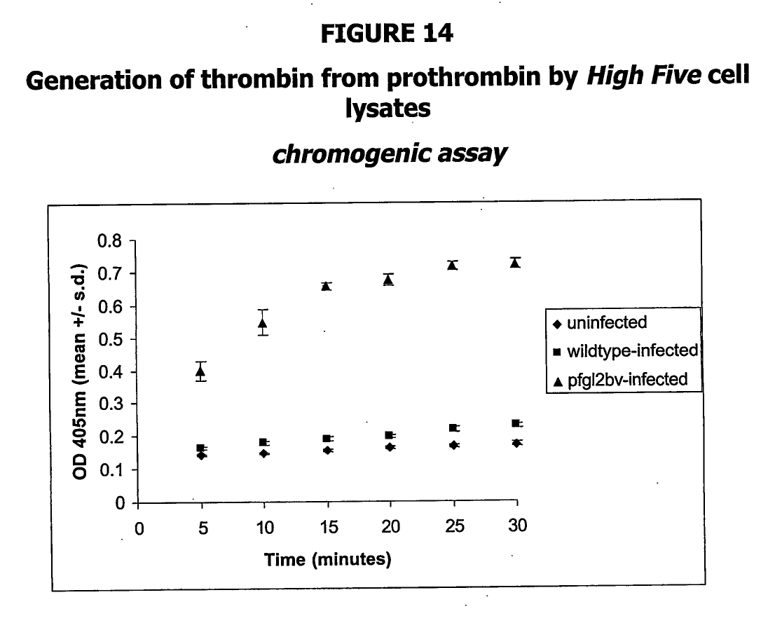

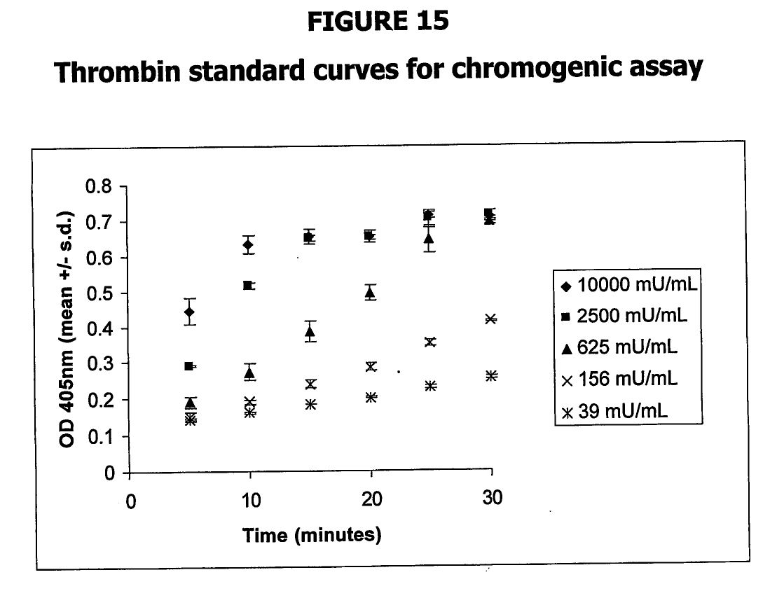

High-Five cells were harvested after three days of infection (MOI 3) by either pfgl2bv or wild-type baculovirus; uninfected cells were harvested in parallel. Cell pellets were washed twice with reaction buffer (20 mM HEPES, 150 mM NaCl, 5 mM CaCl₂, pH 7.4) and resuspended in this buffer at a final concentration of 3×10⁷ cells/mL. Cells were lysed by three cycles of freezing in liquid nitrogen, thawing at 37° C., and vigorous vortexing. Lysates from each group were tested in triplicate. For each individual reaction, the lysate of 3×10⁵ cells (10 ml) was incubated with 10 μl of 20 μM human prothrombin (final concentration 10 μM) at 37° for 60 minutes. The reaction was subsequently diluted with 125 μl of ice-cold pH 8.3 buffer containing 50 mM Tris, 227 mM NaCl, 1% bovine serum albumin, and 1% sodium azide. Reactions were centrifuged at 14,000 rpm to pellet debris and the supernatants were transferred to 96-well plates. 15 μl of a chromogenic substrate of human thrombin (Chromozym TH, Roche) was added to each sample. Thrombin activity in each sample was assayed by measuring the change in absorbance (OD 405 nm) over time in an automated plate reader. A significant level of thrombin activity was generated by pfgl2bv-infected cell lysates in contrast to wild-type baculovirus-infected and uninfected controls (FIG. 14), as judged by comparison with standard curves generated by known concentrations of human thrombin (FIG. 15). No thrombin generation was observed when prothrombin was withheld from the reaction mixture (data not shown). This data suggests that recombinant porcine fgl2 protein is able to generate active thrombin from human prothrombin.

Induction of fgl2 mRNA in Activated Porcine Endothelial Cells

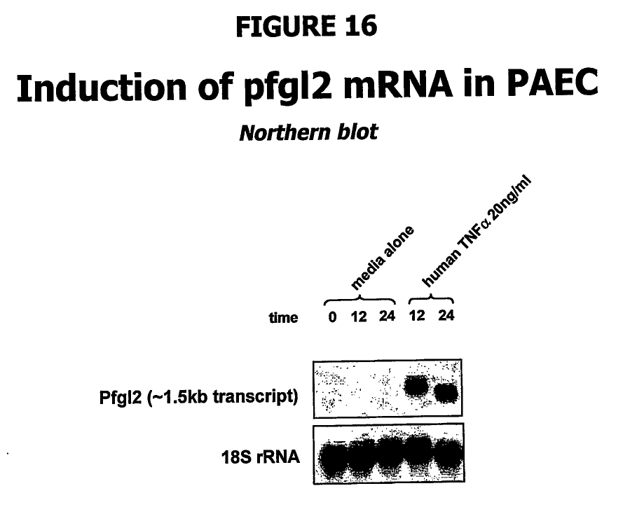

Primary porcine aortic endothelial cells (PAEC) were harvested and propagated as monolayers. PAEC were incubated with 20 ng/ml of human human tumor necrosis factor α (hTNFα). Total RNA was isolated from cells at 0, 12, and 24 hours, and examined for pfgl2 mRNA levels by Northern analysis. 18SrRNA levels were examined as a control for equal loading. Activation of PAEC by hTNFα is associated with induction of pfgl2 mRNA (FIG. 16). This finding suggests that induction of pfgl2 may play a role in the pathogenesis of xenograft thrombosis, a hallmark of which is activation of vascular endothelial cells.

These findings provide preliminary evidence that fgl2 expression is increased in porcine endothelial cells in response to human serum and cytokines that are relevant in xenotransplantation.

### Example 2

Determination of the Chromosomal Location of the Porcine fgl2 Gene Using Fluorescence In-Situ Hybridization (FISH).

Methods

Porcine Lymphocytes

Lymphocytes were isolated from porcine blood using standard techniques, and cultured in a-minimal essential medium (α-NMEM) supplemented with 15% fetal calf serum, 1% L-glutamine, and phytohemagglutinin (PHA) at 37° C. for 72 hours. Cells were harvested and slides were prepared using standard procedures including hypotonic treatment, fixation, and air dry.

Fluorescence In Situ Hybridization (FISH) Assay

The 12.8 kb porcine genomic DNA SalI restriction fragment of genomic library Clone 1 (containing almost all of the pfgl2 gene) was used as a probe for FISH. The DNA was biotinylated with dATP using the Gibco BRL BioNick labelling kit (15° C., 1 hr). The procedure for FISH detection was performed as previously described (51,52). Briefly, slides were baked at 55° C. for 1 hour. After RNase A treatment, the slides were denatured in 70% formamide in 2×SSC for 2 minutes at 70° C., followed by dehydration with ethanol. Probes were denatured at 75° C. for 5 minutes in a hybridization mix consisting of 50% formamide and 10% dextran sulfate. Probes were loaded on the denatured chromosomal slides. After overnight hybridization, slides were washed and detected as well as amplified. FISH signals and the DAPI banding pattern (53) was recorded separately by taking photographs, and the assignment of the FISH mapping data with chromosomal bands was achieved by superimposing FISH signals with DAPI banded chromosomes (52).

Results

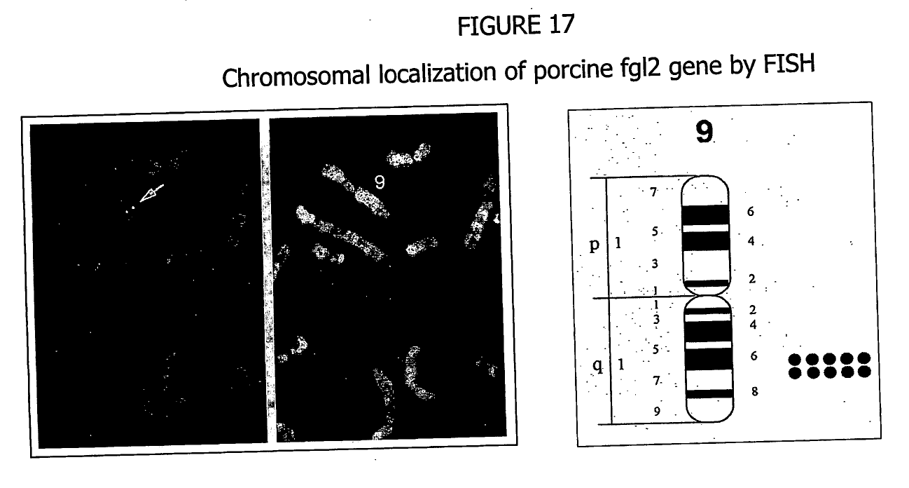

As illustrated in the FIG. 17, the porcine fgl2 probe localized to a single chromosomal locus. The detailed position was determined to be porcine chromosome 9, region q16-q17.

Discussion

This data demonstrates that pfgl2 is present as a single-copy gene in the porcine genome, localizing to 9q16-q17. This chromosomal region is syntenic with human chromosome 7, region q11.23, which is the location of the fgl2 gene within the human genome (55). The latter is syntenic with murine chromosome 5, to which the murine fgl2 gene localizes (54). This data provides further evidence that fgl2 is highly conserved between species not only in gene structure and function, but in its chromosomal location.

### Example 3

Generation of Anti-pfgl2 Antibodies

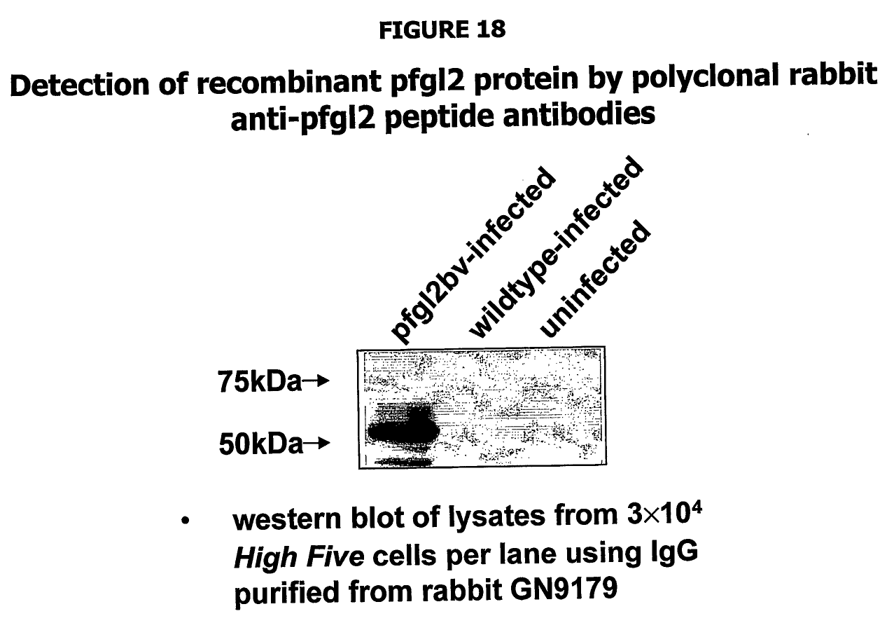

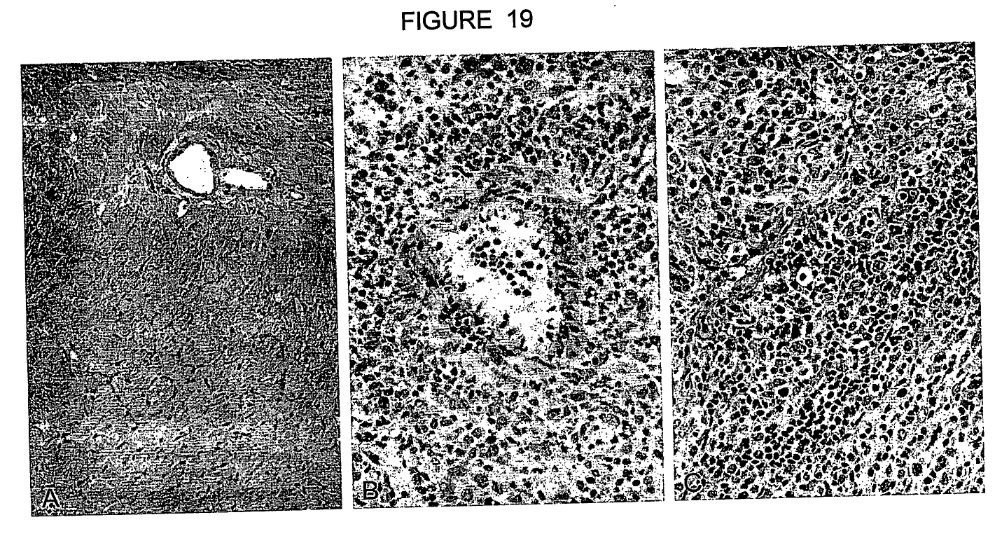

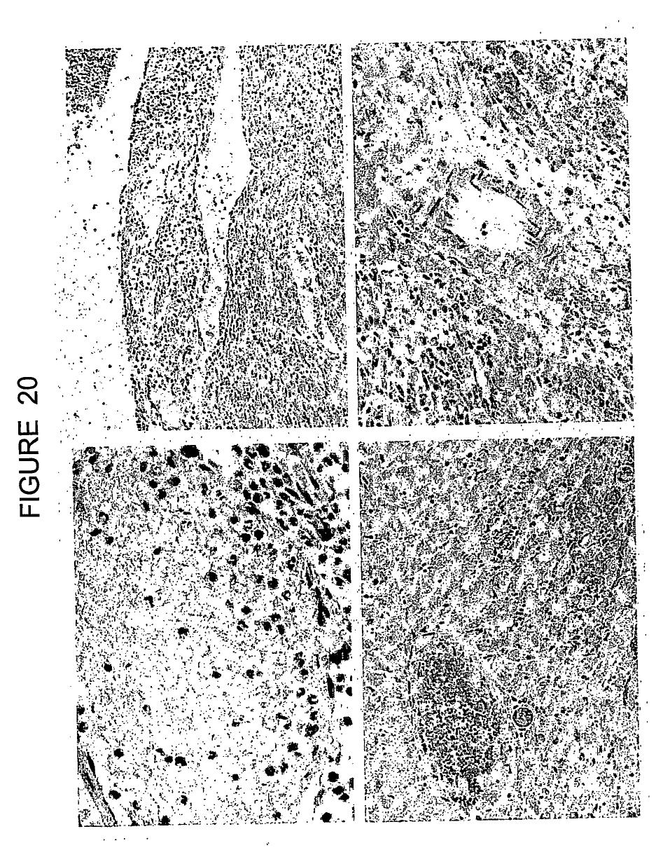

A peptide consisting of the C-terminal 19 amino acids of pfgl2 was synthesized, conjugated to KLH, and used for immunization (by standard protocol) of two New Zealand white rabbits (rabbits GN9179 and GN9180). Post-immunization serum samples collected from both animals were found to react by ELISA against the 19aa peptide used for immunization, while pre-immune serum samples showed no reactivity. IgG was subsequently isolated from the sera by Protein G Sepharose affinity chromatography. On Western blot, the purified IgG from both animals recognized recombinant pfgl2 protein generated by pfgl2bv infection of insect cells (FIG. 18). These results suggest that these polyclonal rabbit anti-pfgl2 antibodies can be utilized for analysis of pfgl2 protein expression in porcine cells and tissues by Western blot.

### Example 4

Role of fgl2 in Xenograft Rejection

In order to investigate the role of fgl2 in xenograft rejection in vivo, the inventors have established a mouse-to-rat cardiac xenotransplant model. Wild-type mouse hearts transplanted heterotopically into rats developed intravascular thrombosis and other typical features of xenograft rejection in association with increased tissue levels of fgl2 mRNA. Through targeted disruption of the fgl2 gene, fgl2 knockout mice were generated for use as cardiac xenograft donors in this model. In contrast to wild-type mouse hearts, cardiac xenografts from fgl2 knockout mice did not develop thrombosis or any other typical features of xenograft rejection following implantation into immunocompetent rats. Moreover, indefinite survival of fgl2 knockout cardiac xenografts was achieved through immunosuppression of recipient rats with a combination of short-course cobra venom factor and daily cyclosporine. Withdrawal of cyclosporine resulted in a pattern of cellular rejection that was similar to that observed with allogeneic grafts, which could be controlled through the use of conventional immunosuppressive agents.

The above data provides in vivo evidence that thrombosis, normally seen during a xenotransplant, is prevented when the donor organ does not express the fgl2 gene.

### Example 5

Preparation of fgl2 Knockout Pigs

The inventors have demonstrated that the use of fgl2 knockout (fgl2 −/−) donor hearts for mouse-to-rat xenotransplantation abrogates the thrombosis classically associated with delayed xenograft rejection (also known as acute vascular rejection, AVR) in this rodent model. Furthermore, treatment of recipient rats with a combination of cobra venom factor and cyclosporine permits indefinite survival of fgl2 −/− donor hearts. Subsequent withdrawal of cyclosporine immunosuppression results not in AVR/DXR, but in typical allograft-like cellular rejection, suggesting that deficiency of fgl2 is protective against AVR.

These observations provide a strong rationale for investigating the utility of fgl2 knockout donor pigs for the prevention of AVR/DXR in pig-to-primate xenotransplantation. Generation of fgl2 knockout pigs can now be achieved based on the availability of the pfgl2 genomic sequence, pfgl2 chromosomal localization, and the recent development of methods related to genetic modification in pigs. Cloning of pigs by somatic cell nuclear transfer has now been established and reported as a viable technology (57, 58). As an extension of this work, other groups have utilized porcine somatic cells for targeted gene disruption by homologous recombination; nuclear transfer from these genetically modified somatic cells has subsequently been performed in order to generate viable pigs with targeted gene knockouts (59, 60). The production of pigs deficient in galactosyl-α1,3-galactose (α-gal, a major xenoantigen) was recently achieved through the application of these technologies to disrupt the gene that encodes α1,3-galactosyltransferase, the enzyme responsible for the production of α-gal (61).

One method for generating a fgl2 knockout pig can comprise the preparation of a pfgl2 gene-targeting knockout vector containing genomic sequences spanning the 5′ and 3′ flanking regions of pfgl2 (necessary for homologous recombination), but with the first exon of pfgl2 replaced with a lacZ-neo-polyA reporter/selection cassette, in a manner similar to that used for generation of the gene-targeted fgl2 knockout mouse. According to established methods (60), porcine fetal fibroblasts are then transfected with the targeting vector and stably transfected clones selected with a neomycin analogue. Site-specific replacement of the native pfgl2 gene by the targeting vector can be confirmed by PCR screening and Southern blot analysis and appropriate clones isolated for use as donors for nuclear transfer into enucleated in vitro-matured pig oocytes. Offspring generated by this protocol can be genotyped using PCR and Southern blotting to identify the presence of native or disrupted pfgl2 alleles. Transmission of the knockout allele into the germline can be confirmed by mating of heterozygote (pfgl2 +/−) offspring in order to generate homozygous (pfgl2 −/−) pfgl2 protein deficient pigs.

Organs from pfgl2 deficient pigs can be utilized for pig-to-nonhuman primate xenotransplantation using methods described previously (62, 63). Mating of pfgl2 knockout pigs with other genetically modified pig lines (eg. hDAF transgenic or α-gal knockout) can also be conducted and used to evaluate the combined utility of fgl2 deficiency with other genetic modifications in the prevention of AVR/DXR, which remains a major hurdle to successful xenotransplantation.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Baer, N. Canada's organ shortage is severe and getting worse. CMAJ. 157, 179-182 (1997).
2. Dorling, A., Riesbeck, K., Warrens, A. & Lechler, R. Clinical xenotransplantation of solid organs. Lancet 349, 867-871 (1997).
3. Dalmasso, A. P. et al. Mechanism of complement activation in the hyperacute rejection of porcine organs transplanted into primate recipients. Am. J. Pathol. 140, 1157-1166 (1992).
4. Sandrin, M. S. & McKenzie, I. F. C. Gala(1,3)Gal, the major xenoantigen(s) recognised in pigs by human natural antibodies. Immunological Reviews 141, 169-190 (1994).
5. Ryan, U. S. Complement inhibitory therapeutics and xenotransplantation. Nat. Med. 1, 967-968 (1995).
6. Auchincloss, H., Jr. & Sachs, D. H. Xenogeneic transplantation. Annu. Rev. Immunol. 16,433-470 (1998).
7. Cozzi, E. & White, D. J. G. The generation of transgenic pigs as potential organ donors for humans. Nat. Med. 1, 964-966 (1995).
8. Bach, F. H., Winkler, H., Ferran, C., Hancock, W. W. & Robson, S. C. Delayed xenograft rejection. Immunol. Today 17, 379-384 (1996).
9. Buhler, L. et al. Coagulation and thrombotic disorders associated with pig organ and hematopoietic cell transplantation in nonhuman primates. Transplantation 70, 1323-1331 (2000).
10. Robson, S. C., Schulte am, E. J. & Bach, F. H. Factors in xenograft rejection. Ann. N.Y. Acad. Sci. 875,261-276 (1999).
11. Robson, S. C. et al. Role of endothelial cells in transplantation. Int. Arch. Allergy Immunol. 106, 305-322 (1995).
12. Bach, F. H. et al. Endothelial cell activation and thromboregulation during xenograft rejection. Immunological Reviews 141, 5-30 (1994).
13. Palmetshofer, A., Galili, U., Dalmasso, A. P., Robson, S. C. & Bach, F. H. a-galactosyl epitope-mediated activation of porcine aortic endothelial cells: type II activation. Transplantation 65, 971-978 (1998).
14. Saadi, S., Holzknecht, R. A., Patte, C. P., Stern, D. M. & Platt, J. L. Complement-mediated regulation of tissue factor activity in endothelium J. Exp. Med. 182, 1807-1814 (1995).
15. Selvan, R. S., Kapadia, H. B. & Platt, J. L. Complement-induced expression of chemokine genes in endothelium: regulation by IL-1-dependent and -independent mechanisms. J. Immunol. 161, 4388-4395 (1998).
16. Kilgore, K. S., Shen, J. P., Mller, B. F., Ward, P. A. & Warren, J. S. Enhancement by the complement membrane attack complex of tumor necrosis factor-alpha-induced endothehal cell expression of E-selectin and ICAM-1. J. Immunol. 155, 1434-1441 (1995).
17. Goodman, D. J., von Albertini, M., Willson, A., Millan, M. T. & Bach, F. H. Direct activation of porcine endothelial cells by human natural killer cells. Transplantation 61, 763-771 (1996).
18. Lin, Y., Vandeputte, M. & Waer, M. Contribution of activated macrophages to the process of delayed xenograft rejection. Transplantation 64, 1677-1683 (1997).
19. Millan, M. T. et al. Human monocytes activate porcine endothelial cells, resulting in increased E-selectin, interleukin-8, monocyte chemotactic protein-1, and plasminogen activator inhibitor-type-1 expression. Transplantation 63, 421-429 (1997).
20. Bustos, M., Saadi, S. & Platt, J. L. Platelet-mediated activation of endothelial cells: implications for the pathogenesis of transplant rejection. Transplantation 72,509-515 (2001).
21. Kirkiles-Smith, N. C. et al. Endothelial cell activation by tumor necrosis factor elicits human antiporcine cell-mediated rejection responses. Transplant. Proc. 33, 412-413 (2001).
22. Saadi, S., Holzknecht, R. A., Patte, C. P. & Platt, J. L. Endothelial cell activation by pore-forming structures: pivotal role for interleukin-1alpha. Circulation 101, 1867-1873 (2000).
23. Moll, T. et al. Regulation of the tissue factor promoter in endothelial cells. Binding of NF kappa B-, AP-1-, and Sp1-like transcription factors. J. Biol. Chem. 270, 3849-3857 (1995).
24. Anrather, D. et al. Thrombin activates nuclear factor-kappaB and potentiates endothelial cell activation by TNF. J. Immunol. 159, 5620-5628 (1997).
25. Chen, D. et al. Human thrombin and FXa mediate porcine endothelial cell activation; modulation by expression of TFPI-CD4 and hirudin-CD4 fusion proteins. Xenotransplantation 8, 258-265 (2001).
26. Malyguine, A. M. et al. Induction of procoagulant function in porcine endothelial cells by human natural killer cells. J. Immunol. 159, 4659-4664 (1997).
27. Jurd, K. M., Gibbs, R. V. & Hunt, B. J. Activation of human prothrombin by porcine aortic endothelial cells—a potential barrier to pig to human xenotransplantation. Blood Coagul. Fibrinolysis 7(3), 336-343. 1996.
28. Siegel, J. B. et al. Xenogeneic endothelial cells activate human prothrombin. Transplantation 64, 888-896 (1997).
29. Fujiwara, I., Nakajima, H., Akioka, K., Matsuda, T. & Oka, T. Soluble complement receptor type 1 and antithrombin-III combination therapy prolongs xenograft survival: the role of thrombin and prostacyclin in hyperacute rejection. Transplant. Proc. 29, 935-937 (1997).
30. Robson, S. C. et al. Thrombin inhibition in an ex vivo model of porcine heart xenograft hyperacute rejection. Transplantation 61, 862-868 (1996).
31. Schwartz, B. S., Levy, G. A., Fair, D. S. & Edgington, T. S. Murine lymphoid procoagulant activity induced by bacterial lipopolysaccharide and immune complexes is a monocyte prothrombinase. J. Exp. Med. 155, 1464-1479 (1982).
32. Dindzans, V. J., MacPhee, P. J., Fung, L. S., Leibowitz, J. L. & Levy, G. A. The immune response to mouse hepatitis virus: expression of monocyte procoagulant activity and plasminogen activator during infection in vivo. J. Immunol. 135, 4189-4197 (1985).
33. Li, C. et al. Monoclonal antiprothrombinase (3D4.3) prevents mortality from murine hepatitis virus (MHV-3) infection. J. Exp. Med. 176, 689-697 (1992).
34. Fung, L. S. et al. Monoclonal antibody analysis of a unique macrophage procoagulant activity induced by murine hepatitis virus strain 3 infection. J. Biol. Chem. 266, 1789-1795 (1991).
35. Parr, R. L. et al. Association of mouse fibrinogen-like protein with murine hepatitis virus-induced prothrombinase activity. J. Virol. 69, 5033-5038 (1995).
36. Koyama, T., Hall, L. R., Haser, W. G., Tonegawa, S. & Saito, H. Structure of a cytotoxic T-lymphocyte-specific gene shows a strong homology to fibrinogen beta and gamma chains. Proc. Natl. Acad. Sci. U.S. A 84, 1609-1613 (1987).
37. Lafuse, W. P., Castle, L., Brown, D. & Zwilling, B. S. The cytotoxic T lymphocyte gene FIBLP with homology to fibrinogen beta and gamma subunits is also induced in mouse macrophages by IFN-gamma. Cell Immunol. 163, 187-190 (1995).
38. Marazzi, S. et al. Characterization of human fibroleukin, a fibrinogen-like protein secreted by T lymphocytes. J. Immunol. 161, 138-147 (1998).
39. Ruegg, C. & Pytela, R. Sequence of a human transcript expressed in T-lymphocytes and encoding a fibrinogen-like protein. Gene 160, 257-262 (1995).
40. Levy, G. A. et al. Molecular and functional analysis of the human prothrombinase gene (HFGL2) and its role in viral hepatitis. Am. J. Pathol. 156, 1217-1225 (2000).
41. Yuwaraj, S., Ding, J., Liu, M., Marsden, P. A. & Levy, G. A. Genomic Characterization, Localization, and Functional Expression of FGL2, the Human Gene Encoding Fibroleukin: A Novel Human Procoagulant. Genomics 71,330-338 (2001).
42. Clark, D. A. et al. The emerging role of immunoregulation of fibrinogen-related procoagulant fgl2 in the success or spontaneous abortion of early pregnancy in mice and humans. Am. J. Reprod. Immunol. 42, 37-43 (1999).
43. Clark, D. A., Chaouat, G., Arck, P. C., Mittruecker, H. W. & Levy, G. A. Cytokine-dependent abortion in CBA×DBA/2 mice is mediated by the procoagulant fgl2 prothrombinase. J. Immunol. 160, 545-549 (1998).
44. Clark, D. A., Ding, J. W., Yu, G., Levy, G. A. & Gorczynski, R. M. fgl2 prothrombinase expression in mouse trophoblast and decidua triggers abortion but may be countered by OX-2. Mol. Hum. Reprod. 7, 185-194 (2001).
45. Halloran, P. F., Aprile, M. A., Haddad, G. J. & Robinette, M. A. Procoagulant activity in renal transplant recipients. Transplantation 39, 374-377 (1985).
46. Cole, E. H., Cardella, C. J., Schulman, J. & Levy, G. A. Monocyte procoagulant activity and plasminogen activator. Role in human renal allograft rejection. Transplantation 40,363-371 (1985).
47. Silverman, R. et al. Immune responses in small intestinal transplantation in the rat: correlation of histopathology and monocyte procoagulant activity. Surgery 102, 395-401 (1987).
48. Kim, P. C. W., Levy, G. A., Craig, M., Cullen, J. & Cohen, Z. Immune responses during small-intestinal allograft rejection: correlation between procoagulant activity and histopathology. Transplant. Proc. 22, 2477-2479 (1990).
49. Smiley, S. T., Gao, W., King, J. A. & Hancock, W. W. Molecular, protein and functional evidence that a cytokine-induced prothrombinase, fgl2, promotes fibrin deposition during allograft rejection. Abstract presented at meeting of the American Society of Transplantation, 2000.
50. Mendicino, M. et al. The involvement of the novel immune coagulant FGL-2 in thrombosis in acute vascular xenograft rejection (AVR). Xenotransplantation 8, 44-45 (2001).
51. Heng H H Q, Squire J, Tsui L C. High resolution mapping of mammalian genes by in situ hybridization to free chromatin. Proc Natl Acad Sci USA 1992; 89: 9509-9513.
52. Heng H H Q, Tsui L C. Modes of DAPI banding and simultaneous in situ hybridization. Chromosoma 1993; 102: 325-332.
53. Lin C C, Biederman B M, Jamro H K, Hawthorne A B, Church R B. Porcine (Sus scrofa domestica) chromosome identification and suggested nomenclature. Can J Genet Cytol 1980; 22: 103-116.
54. Qureshi S T, Clermont S, Leibowitz J, Fung L S, Levy G, Malo D. Mouse hepatitis virus-3 induced prothrombinase (Fgl2) maps to proximal chromosome 5. Genomics 1995; 29: 307-309.
55. Yuwaraj S, Ding J, Liu M, Marsden P A, Levy G A. Genomic Characterization, Localization, and Functional Expression of FGL2, the Human Gene Encoding Fibroleukin: A Novel Human Procoagulant. Genomics 2001; 71: 330-338.
56. Martin et al. Production of transgenic swine, Transgenic Animal Technology: A Laboratory Handbook, Carl A. Pinkert, ed., Academic Press; 315-388. 1994.
57. Polejaeva I A, Chen S H; Vaught T D, Page R L, Mullins J, Ball S, Dai Y, Boone J, Walker S, Ayares D L, Colman A, Campbell K H. Cloned pigs produced by nuclear transfer from adult somatic cells. Nature 2000; 407: 86-90.
58. Onishi A, Iwamoto M, Akita T, Mikawa S, Takeda K, Awata T, Hanada H, Perry A C. Pig cloning by microinjection of fetal fibroblast nuclei. Science 2000; 289: 1188-1190.
59. Lai L, Kolber-Simonds D, Park K W, Cheong H T, Greenstein J L, Im G S, Samuel M, Bonk A, Rieke A, Day B N, Murphy C N, Carter D B, Hawley R J, Prather R S. Production of alpha-1,3-galactosyltransferase knockout pigs by nuclear transfer cloning. Science 2002; 295: 1089-1092.
60. Dai Y, Vaught T D, Boone J, Chen S H, Phelps C J, Ball S, Monahan J A, Jobst P M, McCreath K J, Lamborn A E, Cowell-Lucero J L, Wells K D, Colman A, Polejaeva I A, Ayares D L. Targeted disruption of the alpha1, 3-galactosyltransferase gene in cloned pigs. Nat Biotechnol 2002; 20: 251-255.
61. Phelps C J, Koike C, Vaught T D, Boone J. Wells K D, Chen S H, Ball S, Specht S M, Polejaeva I A, Monahan J A, Jobst P M, Sharma S B, Lamborn A E, Garst A S, Moore M, Demetris A J, Rudert W A, Bottino R, Bertera S, Trucco M, Starzl T E, Dai Y, Ayares D L. Production of alpha 1,3-galactosyltransferase-deficient pigs. Science 2003; 299: 411-414.
62. Ghanekar A, Lajoie G, Luo Y, Yang H, Choi J, Garcia B, Cole E H, Greig P D, Cattral M S, Phillips M J, Cardella C J, Levy G A, Zhong R, Grant D R. Improvement in rejection of human decay accelerating factor transgenic pig-to-primate renal xenografts with administration of rabbit antithymocyte serum. Transplantation 2002; 74: 28-35.
63. Zhong R, Luo Y, Yang H, Garcia B, Ghanekar A, Luke P, Chakrabarti S, Lajoie G, Phillips M J, Katopodis A G, Duthaler R O, Cattral M, Wall W. Jevnikar A, Bailey M, Levy G A, Grant D R. Improvement in human decay accelerating factor transgenic porcine kidney xenograft rejection with intravenous administration of gas914, a polymeric form of alphaGAL. Transplantation 2003; 75: 10-19.

1. A method of inhibiting or suppressing an immune response to a porcine organ or tissue comprising administering an effective amount of an agent that inhibits porcine fql2 to the porcine organ or tissue, porcine donor or transplant recipient.

2. A method of inhibiting or preventing thrombosis associated with xenotransplant rejection of a porcine organ or tissue comprising administering an effective amount of an agent that inhibits porcine fgl2 to the porcine organ or tissue, porcine donor or transplant recipient.

3. A method according to claim 1 wherein the agent is an antibody that binds to porcine fgl2.

4. A method according to claim 1 wherein the agent is an antisense oligonucleotide that is complementary to the porcine fgl2 sequence.

5. A method according to claim 1 wherein the agent inhibits the porcine fgl2 having a nucleic acid sequence shown in FIG. 1A (SEQ ID NO:1) or a homolog or analog thereof or inhibits a porcine fgl2 protein having an amino acid sequence show in FIG. 1B (SEQ ID NO:2) or an analog, homolog or fragment thereof.

6. A method according to claim 1 wherein the organ or tissue is from a transgenic pig lacking expression of the porcine fgl2 gene.

7. A method of modulating an immune response comprising administering an effective amount of a porcine fgl2 nucleic acid sequence, a porcine fgl2 protein or a porcine fgl2 modulator to an animal in need thereof.

8. A method according to claim 7 to modulate an immune response involved in graft rejection.

9. A method according to claim 7 to modulate an immune response involved in fetal loss.

10. A method according to claim 7 to modulate an immune response involved in a viral infection.

11. A method according to claim 7 to modulate an immune response involved in a hepatitis-like disease.

12. A method according to claim 7 wherein the porcine fgl2 has the nucleic acid seqeuence shown in FIG. 1A (SEQ ID NO:1) or a homolog or analog thereof or an amino acid sequence shown in FIG. 1B (SEQ ID NO:2) or an analog, homolog or fragment thereof.

13. A method according to claim 7 wherein the porcine fgl2 modulator is an antibody that binds to fgl2.

14. A method according to claim 7 wherein the porcine fgl2 modulator is an antisense oligonucleotide that is complementary to the porcine fgl2 sequence.

15. An isolated porcine fgl2 nucleic acid molecule having a nucleic acid sequence shown in FIG. 1A (SEQ ID NO:1) or a homolog or analog thereof.

16. An isolated porcine fgl2 nucleic acid molecule according to claim 15 wherein the nucleic acid sequence comprises:
(a) a nucleic acid sequence as shown in FIG. 1A (SEQ ID NO:1), wherein T can also be U;
(b) a nucleic acid sequence that is complimentary to a nucleic acid sequence of (a);
(c) a nucleic acid sequence that has substantial sequence homology to a nucleic acid sequence of (a) or (b);
(d) a nucleic acid sequence that is an analog of a nucleic acid sequence of (a), (b) or (c); or
(e) a nucleic acid sequence that hybridizes to a nucleic acid sequence of (a), (b), (c) or (d) under stringent hybridization conditions.

17. An isolated porcine fgl2 protein having an amino acid sequence shown in FIG. 1B (SEQ ID NO:2) or an analog, homolog or fragment thereof.

18. An antibody that binds to an isolated protein according to claim 17.

19. An antisense oligonucleotide that is complementary to the porcine fgl2 sequence of claim 15.


SEQUENCE LISTING (7 sequences)

SEQ ID NO: 1 - DNA, 8053 bp, Porcine
cagcaagaagttgatccgtgggtggaatccaggtctccagagtgtaaagtaagcctgacacatttagagattatgaatagttaagtttgaataggtcagaggatgtcaacagtttggctgggctagagaagtatttagaaaaatagtaggaaatcaggttaaagaaataaataggagacagattacagggtgctttaaataatataagaataataagaatttggatttttagccatacttttgaaattaagcaataaaatccctttttaaataaaaatcttatatatgagacaccaatatgcaaaacaaattaaaaggaggtggtttctccaattgaagctgttccctctccctgccctcagcctctcaggagaaaagctggaaaaccctaaagagaaagtttgacaagtgtgaacagtcatgctgcagacatgtgtggggcaaggtagtgatacagcaagtggcacacagtccaggtagagggatgtacgctgaaggcagaaagcacagagaggggaccaccaggctgccagtgggaagggaaggcaaagatgctggacacaaaaagaatcagaaagagaggggagtagttcaaagactcacattcagcggccgtggaaggacagtgcaatcacaaaaatagtgaaaggcaaaggaggaggctgcctttgtaaggcagtgatgtgcttttatgtgtttctgtcatctagacatttgtgctgcactatgctgtagtcaaagggatagaaagtagcacttttttctctatcagttttcttctgatctcaaatagatgaaacaaagtctactgcaagaggcagaaaaagctgaacttcgttacatttaaaattgtaagtgcctgagagtctgtctgaaagcccaatgagaagaaaatacaggattcctaatgcccccttccagaaaggaaaaagaaccatttccatttctgcaatatttctgttcctttgcacaatccggataattagcacatttatttttatctttaaaaactcttgcccccactgtggagttaagtacaatggaatgattcatttcacgtataattattttaaaacccaaggttcttcttgtgcttttaaaaaataaacgatgagtgaaacgtgttgaatatgtgcctctgagtacaggaattccaaagcggattatttggattgcggatcagtgacagtttttggcaagcctagttggatgaggacagtctccttttcctctgcgtttgtgaaccttgtgatgaaagtgctcccgcccttttctgagaacagaaagcctgactcaggcaggcagctgctgctcttaaaacagctcgagccggcagcctcccctgctggggtgagcgccaccgcagagatgaagctgaagctgtcgaactggtgctggctgagctccgctgtcctcgcggcttatggctttttggtcgtggccaacaatgagacggaggaaattaaagatgaagcagccaaggacgcctgcccggtgagactagaaagcagagggaaatgcgaagaggtaggcgggtgtccctaccaggtgaacctgccccccttgactcttcagctccccaagcagtttggcaggattgaggaggtgttcaaagaggtccagaacctcaaggaaattgtgaacagcctgaagaagacttgccaagactgcagactgcaggctgacgacaaccgagacccaggcaggaatggactgctgtcaccgggcacaggagccccgggagaagctgatgacagcagagttagagaattggagaacgaggttaacaagctgtcctctgacctgaagaatgccaaggaagagatcgacgggcttcagggtcgcctggagaagctgagccttgtaaatatgaacaacatagaaaattacgttgataacaaggtggcaaacctaacgtttgctgtcaatagtttggatggcaaatgttcatcgcggtgtcccagtcaagaacaaatacagtcacgtccgggtatgtataataatgccttcttatcatttgttccggaatgttttatagctagacaatacccataaacattaacctgggaaaataaaggacagattaagtaaattaagccttttatgcatcaaggtagtacaggcaaaactactgtctttgcagatgtgaccacagaaaggttagaagacctaaggaaccaaaaaaaatcataaagagttctgtctccattagaaagcactttgaatttcaaagaccttatgtgctgacgccagaactacacacaaacagagtggagaaggcaaatttcttaacgtgtcattgtgtttaacatttatctcttcactggaatgtctgaaaacaatggagataaggcagtgtttgtggttgtactctggaaatcaggttttggttttttttttttttgtggccgggtgattgctgcttgtgtggcaacaagagacctttgaaagattttagcaagtaatctgtacttcataatcccaaatatctcgtggaaagcaaagactttatagatgaaataaaagcagcttgtaggcttcacatgtgtgcacaactgtttgctttgagatctagtggcacaaaagcattttaaggggagttttgatatgccactaaagttaggaatatgttcttattccttcctctctttcactagaattcaagcatgtgacaatagaacccatcttccccatccagattatctgaattaactttagtaaataaattaactttagtaaaaaaagatcacaaacctaactgtctactctgcacaagaactgctggctgtggcactggttctcccaaaccctggttttccccacatgagttcctagggccattgctgactgtggtcttattacattttggaaaggcatctctgcattagaatttctgcttattgtaagtgtgaggttaaaggatatggcacttgcagcagagatggtattcatttggaacaagtacctaccttatgcgtgctccattcccagggaatctaggagtgaagggagttgggagtttcatggaccactgttgttattactcccagcagcctgtatctaagggtaccctgcacattgcaaggcagaggttcaagtcaggtaacacacctgggtgcttgttagacgggatgcttcatggcagtttgtaatgacaagaacaagagtctactgcctgtttttctactaagattggggcaagtatctgaatgtttgagttctaaatatacatgaatagattaagtgtggctcatagtgtcttctaccaaacttgctttatctagcacctatttttatagaaattatctgggagtaaatgtctgtatttgattcctgcagaaccacagtagctgattattatacttattctgaatacaagtacgtgtggtttccttctagaatccaaacgtgaaacactaaatattttcctggataataaattcagaagaagaatactggtatataattaaattttttaacttgaacttctttaattgtcaagcaagtaaaataaatggaaatgttgatcagtgctgtactttagaattttaatgtatcaattggaaaaatcctataaaatgtcctcaaagtggaggctattagcatctctaggtcaaaggaaacagaagcatgtgtttcctctgcctgcatggaatgcagttccgggagaagtggaatcacctgaccttcagggccaagggatacagtgcttacaaggacaaggtttgcagccaaccagatctgagtttgtgtcctggctctgccactgaaagtttctgtgaagttgggaaagccagtgatctcctttgagcctaaatattctttctcaccagcagaatggtaataatatctacgtcatacagttcacgtgcagaccaagtgtgaaaatgttatcaagtcattggccaaatgcctagagcaactggggagcccttatgaaatattacggaattattgagactattctctttatttcttgctggtgatgtttgctgttcacattatcaatattatcaccaaccaagaaagtgttggagagcttggtctgctttataacaccaggttcttgttctgaggcctttaacaagcatttgccaaataggttcaagtacgtttagcagagccaggtttcattatcaccattgactgagttatcaaactgcttttgtacatctaaagaattctttcaatgcatagtttttattaacaaaatccaatgcctttattttttttttcactccttcagttcaacatcttatatataaagattgctctgaatactacacaataggcaaaaggagcagtgagatctacagagttacaccggatcccaaaaacagtagctttgaagtttactgtgacatggagaccatggggggaggctggacagtgctgcaggcacgtcttgatggaagcaccaacttcaccagaacatggcgagactacaaagttggctttggaaacctcagaagagaattttggttggggaatgataaaattcatcttctgactaagagtaaggatatgattctaagaatagatcttgaggactttaatggtatcaagctctatgccttgtatgatcacttttatgtggccaacgagtttctcaaataccgtctacacattggtaactataacggcaccgctggagatgccttgcgtttcagtaaacattacaaccatgacatgaagtatttcaccaccccagatagagacaacgatcgatatccctctgggaactgtgggctctactacagctcgggttggtggtttgatgcttgtctttctgcaaacttaaatggcaaatattataaccaaaaatacagaggtgttcgaaatgggattttctggggcacctggcctggtataagtgaggcacaacctggtggctacaggtcctctttcaaagaagccaaaatgatgatcagacccaagtactttaagccataaatcactaatgcttattcctctgggtattcatttcctaatagggcaattaattccttcagcactttggaatgtgtttcatactcctcttcatggcttaaaacttatctctgagcaatgggttcttatgctatacaggatttgaaataaagctgaaaaatatgccttgtaaagaagtcctttgttgctgttacactgttatccaaataaacacttgcaagcaaggggaatattgagaattacacattagatttataattctcttactttcttctatttgaaaagtttttattgctcatcttactcactcgaactaaaaataattgcttattcagcaggctaaattttacacgagtaatctgtattttgcctgagacttgaagacatctgaaggcatattactcttttcaggccctaaataactattgggtatttatttgtctctaaataccctattcctcattctgagataaacttactctatttatggcatttacagcattttagttcccaagcaaagggaaatatgtataattaaacctgttcatgtactaatcctgaaatgctaaattttatttaaaatattttaaaatgcgacaatatactgtccctcctaaaagcattcagaaaattaatttaaccttaaagaccatggtttaaaggtaattcattcatagtttataattcctcagatgttttatgataaaaactgctttaacataaaaaccgtcttcctttgctcttttgcataatagttttaaatttaagactgctcactactgtatgagactactggtagattttctttggtaggtatgggtggggaatgaagcacttatttatagactataacactctgaaggccaatgttatatccaaagcaataatatcattaagtgattcatttcattcaaagctaagttgtataggacaaagagataaatctattcacaaaaagcatatgaattttgaaatatatacagaacaatggttcaaacattatatactttaaccctactggtgtatttcttatttttctatcagaagattcaaatatagttttttttaacaaaacaaaaatagaattttacaattcattaattttagaatgcaagtattatatttaaaagattataaaaattgttaaattgcatcattgtaaaaaagggatcacttcagagagaacagcagtattctactgcatgttaaaaaaaaaaaaatgctcttggagttcccgtcatggttcagcggttaacaaacctgactagtatccatggggatgcaggtttgatccctggcctcactcagtgggttaaggattcggtgttgccatgagctgtggtataggtcacagacgtggcttggatctggcattgctgtggctgtggccggcagctatagctccaattggacccctaccctgcaaatgtgccacaggtgcaacccaaaaaaaaaaaaaaaaaaaaaaaaaaacagctctagagtaaaatctgaaagaatacactgtactatgtgagtcatacaaatacattcaatctttaatttctagtgtctgtttctactaacaaagattttaaagtaaattcacaaaaatgcagctttccccattatttgaaaagattaaggaagagagtagctttagggaagaagctaactttttgaatattgccgtatattaaaaagtctaatcaagaagaagcaagatactcttttaaaaatatataacatgcaaagttttcaggcaaacatgatgaaaactatgtttttatgaatactattaaaatactgaagcaagagaaatgcaaatagaattaagtgatgaccaaaatgtaaaataccattgtagacttcaaaggcttcatttctacttaataggataccaaatgtaaatgttgtaactaatttattctatatttctctcttttttcttgtaaaacaagactaagaagattttgatattatataatgtatttcagacaagggtattttcacttactatatataactatatatatatatataagtttaggtatatttctgtgatcaaaagtgcactccttaaaaaatatagttctaaagaaaaatagctcatataagcgagtgtcccctgatttagtgagaaacaggtgtagtttcaggagaatgtggcaaacaattcatttgatcgacataaatactcaaaagtttgtccttccagttttccatcttatttcaaagaatcaaaattttattttccatcatacttttatggcacctttctaataacaaattacgtaacatagaaattcctttttgaaatcatttcgtgtcacttattatgatactagatatattttagactttgatgatctttaacatattgatggaaatagactatatatgagagaatttttttgccttaacacaaccatttactaatatatgataatggtcgtaaatatggcttgactactagcaaatatattattttagcatcttgccttagttccattttaatggctagtattctcttaattaacaaattgtttcaagctttatcatatttgaaaatattggctaaaattattgggtccttagaaatcccaaatggaactggttcactcatcactgaggaaaaaaaaatcaaattttctctgataagtagttatgctccaaatgaaatttattggatggatccaataaataaaggatccatccwataaataaagaacttaaatacacaaagataaaaaaatatccaagtaccaaaatttgctggctcttagttttgaatggctactcaaggaaagggtagaaggtttaaaggtaaaacatgatgcgtaactcgtcacaataagcccttgcttagaattaatattttcctttatgtataaatacgttgatgtttcatgtcttttaaaaacttgtgaaataaaagatttaatcttcaaatctatttcttcactgtgggaacatgggttactggttgcaaaataatttttacgtacttgtctgaaaagttggaaaaggtttacctaactagacaataactttgacacttcgaaggctttaatgtgtcctatttcatcaacaggaagtccgagaggcaaatagaggaacacca

SEQ ID NO: 2 - protein, 442 aa, Porcine
MKLKLSNWCWLSSAVLAAYGFLVVANNETEEIKDEAAKDACPVRLESRGKCEEVGGCPYQVNLPPLTLQLPKQFGRIEEVFKEVQNLKEIVNSLKKTCQDCRLQADDNRDPGRNGLLSPGTGAPGEADDSRVRELENEVNKLSSDLKNAKEEIDGLQGRLEKLSLVNMNNIENYVDNKVANLTFAVNSLDGKCSSRCPSQEQIQSRPVQHLIYKDCSEYYTIGKRSSEIYRVTPDPKNSSFEVYCDMETMGGGWTVLQARLDGSTNFTRTWRDYKVGFGNLRREFWLGNDKIHLLTKSKDMILRIDLEDFNGIKLYALYDHFYVANEFLKYRLHIGNYNGTAGDALRFSKHYNHDMKYFTTPDRDNDRYPSGNCGLYYSSGWWFDACLSANLNGKYYNQKYRGVRNGIFWGTWPGISEAQPGGYRSSFKEAKMMIRPKYFKP

SEQ ID NO: 3 - DNA, 1033 bp, Porcine
ccctctccctgccctcagcctctcaggagaaaagctggaaaaccctaaagagaaagtttgacaagtgtgaacagtcatgctgcagacatgtgtggggcaaggtagtgatacagcaagtggcacacagtccaggtagagggatgtacgctgaaggcagaaagcacagagaggggaccaccaggctgccagtgggaagggaaggcaaagatgctggacacaaaaagaatcagaaagagaggggagtagttcaaagactcacattcagcggccgtggaaggacagtgcaatcacaaaaatagtgaaaggcaaaggaggaggctgcctttgtaaggcagtgatgtgcttttatgtgtttctgtcatctagacatttgtgctgcactatgctgtagtcaaagggatagaaagtagcacttttttctctatcagttttcttctgatctcaaatagatgaaacaaagtctactgcaagaggcagaaaaagctgaacttcgttacatttaaaattgtaagtgcctgagagtctgtctgaaagcccaatgagaagaaaatacaggattcctaatgcccccttccagaaaggaaaaagaaccatttccatttctgcaatatttctgttcctttgcacaatccggataattagcacatttatttttatctttaaaaactcttgcccccactgtggagttaagtacaatggaatgattcatttcacgtataattattttaaaacccaaggttcttcttgtgcttttaaaaaataaacgatgagtgaaacgtgttgaatatgtgcctctgagtacaggaattccaaagcggattatttggattgcggatcagtgacagtttttggcaagcctagttggatgaggacagtctccttttcctctgcgtttgtgaaccttgtgatgaaagtgctcccgcccttttctgagaacagaaagcctgactcaggcaggcagctgctgctcttaaaacagctcgagccggcagcctcccctgctggggtgagcgccaccgcagagatgaagctg

SEQ ID NO: 4 - DNA, 1015 bp, Homo sapiens
tgccttcagcctctgaagagaaagttagaaaactattatcattaatgctacatgttttgaacaagctgatataccaagtggcccagagagcaggtagaagaaccagcgtggagacagaaagcaagaggcccgcctgccagggctacctgcagaaagaaagggcaaagatgctgtaggcaagagaagttcaggacagacactggcatagctcaaagattcacatttgagcagctgtggaagatgacagtacaattaccaaaatgtcgaagggcaaaggaggcagctactggttttgatgaaagacaattatgtccttttaaatgggtcttagacatttagacatttatatacactatgctacggacaaaggaatagaaagtagcacttttttctccactagttttcttctctttttcaagtagatgaagcaaaagtcaactgcaatagtcagaaagctgtactttgttacacttagaaacttctaaaagtgcttaagatttcacctgaaagtccaacatgaagaaaatacaggctccccaatgccccattctaagaaggaaaaaggaccattttcattttagtaacgtttctgttctatagacagtttggataactagctcttactttttatctttaaaaactgtttttccagtgaagttacgtataattatttacttcaagcgtaagtataccaaattactttagaaatgcaagacttttcttatacttcataaaatacattatgaaagtgaatcttgttggctgtgtacatttgactataataatttcaatgcatattatttctattgagagtaagttacagtttttggcaaactgcgtttgatgagggctatctcctcttcctgtgcgtttctaaaacttgtgatgcaaacgctcccaccctttcctgggaacacagaaagcctgactcaggcagctgccgctattaaagcagctccagccctgcgcactccctgctggggtgagcagcactgtaaagatgaagctggctaac

SEQ ID NO: 5 - DNA, 1015 bp, Murine
gaaaagtcttgggaaatctggttagagatataaatatgagaactggacatggtggtacacacctgtgatctctgtgtttaggagggagaggcagagagatcaggagttcaaggccagcctgagctacttgagacccagtctaaataaataagagatagattacagagtgcctttaactagtacagagaaagaatttgggtttatctgtgtcagttacgctgaaataatttttaagtaataaaatcccttttaataagaaaccttatgaggtcagtatgcacaatgaacttaagagagacccccagctcctgagctgagtgatggggaaggacagccactgcctgtgatgtgtgagtgacgtgcttccaagtgttttaaccactgacgattacatagcctgcacagtcaggagaaaacagccgtattctctgccagttctcttcccttttacaaacagatgagagacacacacagagaatccatttaaagagcggacctttgttctgattaggggcaattttaagtacttaagagttcacacaaagtctagccttcaaaaagaaaacaggttcccaaactagggaggaaacagaatcatttccattttggtgacatttagtgggaagaagctcacagacatttagacgttccaactctttccccactagtggaccaagtatataatatggtatcttttgggcactggtattacaactgttttttaaacaaaagactttccttgtgctttactaaaaacccagacggtgaatcttgaatacaatgcgtggcacccacggcaggcattctattgtgcatagttttgactgacaggagatgacagcatttggctggctgcgcttgctgaggaccctctcctcctgtgtggcgtctgagactgtgatgcaaatgcgcccgcccttttctgggaactcagaacgcctgagtcaggcggcggtggctattaaagcgcctggtcaggctgggctgccgcactgcaaggatgaggcttcctggt

SEQ ID NO: 6 - protein, 439 aa, Homo sapiens
MKLANWYWLSSAVLATYGFLVVANNETEEIKDERAKDVCPVRLESRGKCEEAGECPYQVSLPPLTIQLPKQFSRIEEVFKEVQNLKEIVNSLKKSCQDCKLQADDNGDPGRNGLLLPSTGAPGEVGDNRVRELESEVNKLSSELKNAKEEINVLHGRLEKLNLVNMNNIENYVDSKVANLTFVVNSLDGKCSKCPSQEQIQSRPVQHLIYKDCSDYYAIGKRSSETYRVTPDPKNSSFEVYCDMETMGGGWTVLQARLDGSTNFTRTWQDYKAGFGNLRREFWLGNDKIHLLTKSKEMILRIDLEDFNGVELYALYDQFYVANEFLKYRLHVGNYNGTAGDALRFNKHYNHDLKFFTTPDKDNDRYPSGNCGLYYSSGWWFDACLSANLNGKYYHQKYRGVRNGIFWGTWPGVSEAHPGGYKSSFKEAKMMIRPKHFKP

SEQ ID NO: 7 - protein, 432 aa, Murine
MRLPGWLWLSSAVLAACRAVEEHNLTEGLEDASAQAACPARLEGSGRCEGSQCPFQLTLPTLTIQLPRQLGSMEEVLKEVRTLKEAVDSLKKSCQDCKLQADDHRDPGGNGGNGAETAEDSRVQELESQVNKLSSELKNAKDQIQGLQGRLETLHLVNMNNIENYVDNKVANLTVVVNSLDGKCSKCPSQEHMQSQPVQHLIYKDCSDHYVLGRRSSGAYRVTPDHRNSSFEVYCDMETMGGGWTVLQARLDGSTNFTREWKDYKAGFGNLEREFWLGNDKIHLLTKSKEMILRIDLEDFNGLTLYALYDQFYVANEFLKYRLHIGNYNGTAGDALRFSRHYNHDLRFFTTPDRDNDRYPSGNCGLYYSSGWWFDSCLSANLNGKYYHQKYKGVRNGIFWGTWPGINQAQPGGYKSSFKQAKMMIRPKNFKP